SEQUENCE LISTING (18 sequences)

SEQ ID NO: 1 - DNA, 98 bp, Apium graveolens
  source 1..98: mol_type = genomic DNA
taaaagatgttaacatgtgccttcaagtctctttctgcattcttgatggttttagcatttttattaacttcttttttgtgtgtgttacttaatatgta

SEQ ID NO: 2 - DNA, 98 bp, Apium graveolens
  source 1..98: mol_type = genomic DNA
taaaagatgttaacatgtgccttcaagtctctttctgcattcttgatggtattagcatttttattaacttcttttttgtgtgtgttacttaatatgta

SEQ ID NO: 3 - DNA, 101 bp, Apium graveolens
  source 1..101: mol_type = genomic DNA
aaaaagttccaaaacaatcaaacataaaatagattcaacctaccattactaaaaatrtttatgttctatgtcataaagcctcaatattctgaaattacaca

SEQ ID NO: 4 - DNA, 101 bp, Apium graveolens
  source 1..101: mol_type = genomic DNA
aaaaagttccaaaacaatcaaacataaaatagattcaacctaccattacttaaaatrtttatgttctatgtcataaagcctcaatattctgaaattacaca

SEQ ID NO: 5 - DNA, 101 bp, Apium graveolens
  source 1..101: mol_type = genomic DNA
gctgcaaattttccccatggccgccgccttacgcctctatatctcttccagtcttgtggagactgactttcaggtgccacaacctctatttttatgtcctc

SEQ ID NO: 6 - DNA, 101 bp, Apium graveolens
  source 1..101: mol_type = genomic DNA
gctgcaaattttccccatggccgccgccttacgcctctatatctcttccactcttgtggagactgactttcaggtgccacaacctctatttttatgtcctc

SEQ ID NO: 7 - DNA, 9052 bp, Apium graveolens
  source 1..9052: mol_type = genomic DNA
acacaattcacacaaacgcgcacatacactcaggttcgtttttccttctctgcttcctcttcttcaccccccttccatccaattcaatccaatctatacatacacatatacacacaattctcacacgcctacacaattattatacatatatcttcactctattcactcaatttcaccaaaaagttttgatctttaatcaattaagcatctttaattacatgtatatatgtttgtgtgtatggattgattgtaatgtgtttgaatttttgttgattatgtgattaatgaattagatctgtgtttttggtgcagaaaattgttgatttgaataagattggaggtgattagggtttggatttatgtccgtcaatcaatctagggctgagaagagcgagtcgggacagcatcgaaaaactaatcgatccggtaatagctccggtgggcagaggaattttccaggcggtggtggtaagggtagcggtgctgccaccgcccctactccttcgctttcgactagtcgaaggtatattaggtttcctgtctttgttttttgtaattgtgtgtacatttagacctcgataaattattatgcaatttgttagttgtctaagattaattggtactcggttggttagtaatctcgataatttgatacgtgattggttagtgacctaagattgttggtacttggttagtaatcttgacaatttgatacgctattggttagtaacctaagattgttggtacttggttggttagtaatctcgataattaaatactacgtgattggttagttagctaataagtgcttgattggtttgtagtctttataaattaatatgtgggggtgattggtgacctaagattgtttagtgcttgattggataaattcagtacgtgattattggttatatactaagattaaagaatgtgttttttttaatttatcaaggatacttaggtttgttttacctgtagttaatttgtaagctttattaattaagtaatagaatctttggtccggacagtatgaagttatagagtatacttgtctgttttgtgtacttttctgatgattctaatgattttttttttaattttttagttttaagaagcctagtaatgcgcaaggatcgcaaattcgggttgtgaattcggatattaggaatgatgctactgccggtggtaatgttgctgtttctggttcagcccgtggtgtgcagaatggcgctcattcacaatcacatggtattgaaagaatttttgttcaaggattttaattgtttttgtttttgaatgtggtgtgatggtcaagtatggttttttattttgttgtcgttgtaggagggtctgatgctccagctgttgtcaagcagaatgttactccgacaccaaaaattaatcggggtgtgccgaaagctcctgctccaaataatacccccgtaagctcaaatacaatggccccttcaacccctgtcaaaggttgagtctcgttctatgtaaatatttggcattgtacatgatgaagttaaatatatgaacttctgtatttcataataagctaattggagttttttgtggtcgtggctcaggtggaggttttcctctccagtttggatcaataagtcctggtctgatgcaggtagggctgaaagttttcatcaagaatgtatattgttagctatctcagttacctgaagcttaagaatgatattggtcttcatataattataattattggaatgatttcggttgtggtttaggtacctgctcgaactagctcagcgccgccaaatttggatgagcagaaacgggaacaggttttaaacattgttgccatctttttttgttcattgtgatgtgggggtggggcttgcaaataggatttacttgcaatttctagatgaatatttttaatatcagtattctcttattatgatgggaggctaaatcatttgtaactttgatttctaaatggttgatgtgaagtatattacttatgatccctctggtccaaatagataggctacattttgtggtgtcccacatttgttgaatctctattttttcacgacacacttgaaattccaatctagtttttgagtaatctattatagaggatgaaaaagggtttggttaattgtaatatgatgtgctttctgtagaatgacttttggccctagtgcaatgagatgagttgattaaattacagaataggctgattatgctctgctctttatccctgttgatattaattgtttgacaacttcctttcctgcttgtcaattatttttgcaggctcgctatgaatcatcaagagttatgcctgtgctgccagcccaatccgttcctaagcagaatgtgcctaccaataattctgcaacttctgaccaacctaatattgttgacactcattcgatgtcaaaagcaagaagagatgtgcaagttgcacctggtccacatgcaatccaaactcagaagccttctgttcaccctatttctgggatgcctatgcaaatgccatttcaccagcaacatataccagttcaatttgggaatcctaatccacagttgcagtcccaaagcatggtaaatagctctatgccaatgccaatgggaatgcccttccctatgggaaatccttctcaagtgcagcagcaactgtatattcagggtcttccaccccacatgatgccacctcagggtgtcatgcatcagggtcagggtgtcaatttctcctcgcaaatgggtacccagttacctcacatggggaatatggggatgaacatcaatccccagttctcgcagcagcagccaggaaattttggtggtgctcgtaaaacagttaagataactcaccctgatactcatgaagagttgagtttatctaaaaggactgatacttacaaagaatctggatcgtctgctccgcgatcacatacgaatataccacctcagtcccaacctatttcatcttttcctcctggccgtccaagtaacttttaccctaattcttacagtcagggttccgtattttatacaggtcctaattctcttcatttaagcagtaaccaagttcctccaaattcacaggctccaaggttatataaacaggttagcctgtctttattaaaccgtattaccgtgttgttttatccactgctggtattttgttgcatgccattactgaaatgttatttacggtacatgctcatttaaaatacaggtcaccgtgaagccagcagcttccccacgcttggaaaaaattgctgaaacttctttgccagcaagctcacctactgttgagaaaaatattcctaagatatctggtcaagaaggagaagctacatctattcgtactgaaagagatcctgaaaagacaagtgatatgtcctcacagaagtcagagtatacatcgatacctgttcaaattaaaggaactggcggagtatctgattcagcttccgcatctagccctctaccaagttctattccttgtacacctcctgaaaattccgagtattccacaggctctagtattactgtagttgcaaaaaatgatctattggataggtctaatgataatgatgagaaacacagtaagaagagccaatctgtactgcagaatcaggtattaccctctttttaagattttttattccagcagtatacttcagttgctgtagtgtatggttttaacaaattttgttctcagattgatcgacctgttgccccttcgttacccgaagttgtagaggacacaagatcatcttctataccagaagctcagccaggagaggaaaatgtcagggaatcagaaacctctgttgccgttgacttaaatacaaatagttctgaggtacagggaattcatgagttggaaaaaagcgatggagttgattctgctgaaatgtcagctgatgtatctgtggataataagccaaaacatgaagctatgcgaactcaagaaagtagaaacatactgctctctgagccaaatgcagatgctgctggttgtgtggcaagtgcaaaatccgattcaccaagaccatatgaacatgctgatcaaactgagagccttatcatggaagtgaaagataaacagctgaagttggagatggaacctgtacacctcacaaaaggtgatgcagatgctgatttatcaacctctatagttgggacagtggactgcctgattactgaaacatcctcattgtctcttggatccacaatctcacatgattatgataagacttcagcttcggatgcttctactataatcgataataccatggctagtacagaaacagaatccaacaagttagatcagaaatcacaaattctttcagttccatctcagtctgagccgccttgtgaaactgaaattgctgatagtgatagcacagaattgctttcttcatcaaatgatttgaaagataaacctgtgccggagacaattatgtcaaagaatacaaatacaaagaagaagagaagagaagctttacaaaaggcagaccgtgctggtagaattgctgatctttatatggcatataaaggtccagatgaaaagaaagaaaatccgatcgatgttgagagctcagaaacgtcttctagccaagctaaagagttggcttctggattttctccgaaagatgttccaaatgagagtaaagctgaaccggatgactgggaagatgctgcagatttatcaactccaaaacttgatgatgggaagctccttggagaggtaaaagatcacattgaggacaaaactttaatggacagaaagtactctagagatttccttcttaaattttcagagcaatgcaaggatcttcctgtgggttttaagatcaccttagatattgcagaagcattggtattgtctattggcaatgttcctttgcccagtccaggaagaaatgatgataggtcaatgggaaggtctccatcaggtcgccgtggtgttggcatgggtatgggcgatgatgataaatggagtaaggtacccgggcctcttccccctggactggatatgggttatggaagtcatgcaaacaatgtttttcaacctggagcaaattttggagtattgagaaatccacgtgttcaatcaccagttatgcattccagtggaatcctatctgggccagttcattctatgggtcctcagtatggaatgcagcgaactaactctgatgccgacaaatggcagcgggcgactaattttcagagaggtctaatgccttctcctcggactcccgcacaggtgatgcataaggctgagagaaagtatgaagtgggtaaaataactgacgaggaacaggcaaagcaaaggcagttgaaagctattctgaataagttaacaccgcaaaactttgaaagactatttgagcaagtcaagcaagtaaatattgacaatgctggtactctcactggtgtgatatctcaaatctttgataaagctcttatggaaccgacattttgtgagatgtatgctgatttctgctatcatcttgctggcgacttgcctgattttaatgaagacaacgaaaaaattacatttaagaggttacttctgaataagtgccaggaggagtttgagagaggtgagagggaacaagaagaagccaacagagctgaagaggaaggtgaggttaaacaatctgatgaagaaagggaagagaagagagtccaggcacgaaggcgaatgttgggcaacatcagacttattggggaattatacaagaaaaagatgttaacagagagaattatgcatgaatgcattaagaaactattgggccagtaccaaaatcctgatgaggaagatgtagaagccttgtgcaaactaatgagcacaataggagagatgatagatcaccctaaagccaaggagcacatggatgcatattttgatatgatggccaagctatctaataacatgaagctctcttcgagagtgaggttcatgctgaaggattcaatggacctcaggaaaaacaagtggcagcagaggaggaaagttgaaggtccaaaaaagatcgaggaagtgcacagggatgctgcgaatgagaggcaggcacaagctaacagattggctcgtggaccgagtatgggttcatcttttagaaggggtcaacaacccatggactttgctccaagagggtcaaatgtattatcatcgccaaattcccatatgggaggattccgtggagcaccacaacaaccacgcggttatgccactcaagatattaggacagatgagagacacccttttgataacaggaacctattgccacaaagaccccttggtgactccattacccttgtgccccaaggtggtcttgccagaggaatgtctattagaggacagcccccaatgttaagcgtacccttctctgacatgcacaatcaagattccagaaggactacccctggtctcaatgggtacggttctgtaacagaccggccagtttatacctcaagagaggaactcattccgcgatctgctcagagatttgtcagcccgtctgctcatgaccacatgaatataacggatggaaattcgacttatatgaacagggaaatgccgaatccagatcgtgtttttaacagatctcggcctaatacgcctccttccagaggtatggagtctatttctgtaggaaacattcctacggaaaaggtgttgccggaagaacaattgcgaaaaaagtcattggaaactattaaagaattctacaggtatgatgagaccttgttgcttgcttgtggttgttctttttaattatctttacaactaaaaagctaattttaattgcccttttattccgtgtgctgggtatgatagaaattaacatatcatatatgctagttttacaagttcattgttttttaagtctgagatttgatggggtgaatgtgtattttgtctcttagtggtcttgcaacaatatgaaactgtgtactttatatctgctctttttagtgtatttgcattttatcttgctgtcattaggcgcccagcagatcatcggctattgctatcaataaaaacagtcaaagaatgtgaatcttagaattttgtgtttacccttgtgtttctaggtgtagtatttttgaatcttgatgcacctttactaattgacggttatagtttagaaggattaaagatactagcaagtcgagagagagctaacatttatatattttacaaaataaacattaattttaaagttaaccctgtgtaattcgaaatatacctaaatactgtttatatctgatttctaggcctttagtagagcctcagttgtcattctatgtagtcaaatagttcatttttggagttatcaatgaattttggtatgaaaatacaggaaaagtaaaatgattattgtaaataaggtcagccagtatacataatgattgctactgtgaagaaatgcagtataatattatattctatgttgcttccttccactatacctttcgtgagagtccatcttttcaaaatttagtttacccatcaacttatatgcacacataggagaggctttttcttgcatcactgtttgtgtacagcttaaaatggtcttaggtctttgttgtcttgtattcctagtactctatgagtccatctattgttaccacttattattagtcaattcattgatggctggctgttcccttctttgagtggtgggaattagtcaaacgaaatttcctgtacaataagcctaatgagattggtgtacatgatagattttgaacttactaggttttgaattctcccttcaaaattaaggagcatgtttctatttaggattggcttccttctccatttgctattcagattgaggtccctttctagttaatcattgctttcagccatggccgtagtattagtctgatttaggccttgtactcctgaaacttgtcacctgggcaagtgtcaatatttgatccggcttgttactaggccctctcccaggctcttttgactatttgatctggcttgttacgcagccgtagcccaggttttttcatataggattcttcatctagaataacaggcatttgctttgtttgcgaaagttgtgatgagttgattaaagtattttgagatacagccctgcttcctctttcagttttactgtatattgactcaaaagatgctcttaaatctcagtgccaaagacgagatggaagttgccctgtgtgtcaaagatttgaatgctcccagcttttatccatcaatgatatccatctggataactgattccttcgagaggaaggacatggagaggggtctacttgctaagcttctggtcaaccttgcaaaatctcgggacaccttgttaagtcgacctcaatttgtagaagggtatgtaatggttacctgattcttgcttacaccacttcactgactaagttttatcctagaaggtcaaaatatgttgtccggtgttctttcacttcataattgtttttttgccaggtttgaatctgttctagcttgtttggaggatgctgtaactgatgctcctaaagcaccagaatttcttggtggtatctttgctaaatttgtgctggaaaatgtgcttcccatggcagaaattggaaggttaatttatgaaggtggggaacaacaaggacagcttgtagaaatagggcttgcagctgaagttcttggaagcgtcctggagattattaaaactgaaaaaggagaacaggtcttgaaagaaatttgcacgggctctagtttacgtctagaaaactttcggcctccgaactctaaaagggcattgacgctagataaatttatctaagtgtagccatgattattgtaatctgcatagaagaaattttatgtccagccctatcagtgaatctcttcatattgcagatatatttatctctagatggtcacttgggttgggttttataaatatagccctcttcatattatatatatatatatatatatatattatccagaatcccaaacatccatggtttttaatgaacatccaaaaatttcagtttatcaaca

SEQ ID NO: 8 - DNA, 5328 bp, Apium graveolens
  source 1..5328: mol_type = other DNA
atgtccgtcaatcaatctagggctgagaagagcgagtcgggacagcatcgaaaaactaatcgatccggtaatagctccggtgggcagaggaattttccaggcggtggtggtaagggtagcggtgctgccaccgcccctactccttcgctttcgactagtcgaagttttaagaagcctagtaatgcgcaaggatcgcaaattcgggttgtgaattcggatattaggaatgatgctactgccggtggtaatgttgctgtttctggttcagcccgtggtgtgcagaatggcgctcattcacaatcacatggagggtctgatgctccagctgttgtcaagcagaatgttactccgacaccaaaaattaatcggggtgtgccgaaagctcctgctccaaataatacccccgtaagctcaaatacaatggccccttcaacccctgtcaaaggtggaggttttcctctccagtttggatcaataagtcctggtctgatgcaggtacctgctcgaactagctcagcgccgccaaatttggatgagcagaaacgggaacaggctcgctatgaatcatcaagagttatgcctgtgctgccagcccaatccgttcctaagcagaatgtgcctaccaataattctgcaacttctgaccaacctaatattgttgacactcattcgatgtcaaaagcaagaagagatgtgcaagttgcacctggtccacatgcaatccaaactcagaagccttctgttcaccctatttctgggatgcctatgcaaatgccatttcaccagcaacatataccagttcaatttgggaatcctaatccacagttgcagtcccaaagcatggtaaatagctctatgccaatgccaatgggaatgcccttccctatgggaaatccttctcaagtgcagcagcaactgtatattcagggtcttccaccccacatgatgccacctcagggtgtcatgcatcagggtcagggtgtcaatttctcctcgcaaatgggtacccagttacctcacatggggaatatggggatgaacatcaatccccagttctcgcagcagcagccaggaaattttggtggtgctcgtaaaacagttaagataactcaccctgatactcatgaagagttgagtttatctaaaaggactgatacttacaaagaatctggatcgtctgctccgcgatcacatacgaatataccacctcagtcccaacctatttcatcttttcctcctggccgtccaagtaacttttaccctaattcttacagtcagggttccgtattttatacaggtcctaattctcttcatttaagcagtaaccaagttcctccaaattcacaggctccaaggttatataaacaggtcaccgtgaagccagcagcttccccacgcttggaaaaaattgctgaaacttctttgccagcaagctcacctactgttgagaaaaatattcctaagatatctggtcaagaaggagaagctacatctattcgtactgaaagagatcctgaaaagacaagtgatatgtcctcacagaagtcagagtatacatcgatacctgttcaaattaaaggaactggcggagtatctgattcagcttccgcatctagccctctaccaagttctattccttgtacacctcctgaaaattccgagtattccacaggctctagtattactgtagttgcaaaaaatgatctattggataggtctaatgataatgatgagaaacacagtaagaagagccaatctgtactgcagaatcagattgatcgacctgttgccccttcgttacccgaagttgtagaggacacaagatcatcttctataccagaagctcagccaggagaggaaaatgtcagggaatcagaaacctctgttgccgttgacttaaatacaaatagttctgaggtacagggaattcatgagttggaaaaaagcgatggagttgattctgctgaaatgtcagctgatgtatctgtggataataagccaaaacatgaagctatgcgaactcaagaaagtagaaacatactgctctctgagccaaatgcagatgctgctggttgtgtggcaagtgcaaaatccgattcaccaagaccatatgaacatgctgatcaaactgagagccttatcatggaagtgaaagataaacagctgaagttggagatggaacctgtacacctcacaaaaggtgatgcagatgctgatttatcaacctctatagttgggacagtggactgcctgattactgaaacatcctcattgtctcttggatccacaatctcacatgattatgataagacttcagcttcggatgcttctactataatcgataataccatggctagtacagaaacagaatccaacaagttagatcagaaatcacaaattctttcagttccatctcagtctgagccgccttgtgaaactgaaattgctgatagtgatagcacagaattgctttcttcatcaaatgatttgaaagataaacctgtgccggagacaattatgtcaaagaatacaaatacaaagaagaagagaagagaagctttacaaaaggcagaccgtgctggtagaattgctgatctttatatggcatataaaggtccagatgaaaagaaagaaaatccgatcgatgttgagagctcagaaacgtcttctagccaagctaaagagttggcttctggattttctccgaaagatgttccaaatgagagtaaagctgaaccggatgactgggaagatgctgcagatttatcaactccaaaacttgatgatgggaagctccttggagaggtaaaagatcacattgaggacaaaactttaatggacagaaagtactctagagatttccttcttaaattttcagagcaatgcaaggatcttcctgtgggttttaagatcaccttagatattgcagaagcattggtattgtctattggcaatgttcctttgcccagtccaggaagaaatgatgataggtcaatgggaaggtctccatcaggtcgccgtggtgttggcatgggtatgggcgatgatgataaatggagtaaggtacccgggcctcttccccctggactggatatgggttatggaagtcatgcaaacaatgtttttcaacctggagcaaattttggagtattgagaaatccacgtgttcaatcaccagttatgcattccagtggaatcctatctgggccagttcattctatgggtcctcagtatggaatgcagcgaactaactctgatgccgacaaatggcagcgggcgactaattttcagagaggtctaatgccttctcctcggactcccgcacaggtgatgcataaggctgagagaaagtatgaagtgggtaaaataactgacgaggaacaggcaaagcaaaggcagttgaaagctattctgaataagttaacaccgcaaaactttgaaagactatttgagcaagtcaagcaagtaaatattgacaatgctggtactctcactggtgtgatatctcaaatctttgataaagctcttatggaaccgacattttgtgagatgtatgctgatttctgctatcatcttgctggcgacttgcctgattttaatgaagacaacgaaaaaattacatttaagaggttacttctgaataagtgccaggaggagtttgagagaggtgagagggaacaagaagaagccaacagagctgaagaggaaggtgaggttaaacaatctgatgaagaaagggaagagaagagagtccaggcacgaaggcgaatgttgggcaacatcagacttattggggaattatacaagaaaaagatgttaacagagagaattatgcatgaatgcattaagaaactattgggccagtaccaaaatcctgatgaggaagatgtagaagccttgtgcaaactaatgagcacaataggagagatgatagatcaccctaaagccaaggagcacatggatgcatattttgatatgatggccaagctatctaataacatgaagctctcttcgagagtgaggttcatgctgaaggattcaatggacctcaggaaaaacaagtggcagcagaggaggaaagttgaaggtccaaaaaagatcgaggaagtgcacagggatgctgcgaatgagaggcaggcacaagctaacagattggctcgtggaccgagtatgggttcatcttttagaaggggtcaacaacccatggactttgctccaagagggtcaaatgtattatcatcgccaaattcccatatgggaggattccgtggagcaccacaacaaccacgcggttatgccactcaagatattaggacagatgagagacacccttttgataacaggaacctattgccacaaagaccccttggtgactccattacccttgtgccccaaggtggtcttgccagaggaatgtctattagaggacagcccccaatgttaagcgtacccttctctgacatgcacaatcaagattccagaaggactacccctggtctcaatgggtacggttctgtaacagaccggccagtttatacctcaagagaggaactcattccgcgatctgctcagagatttgtcagcccgtctgctcatgaccacatgaatataacggatggaaattcgacttatatgaacagggaaatgccgaatccagatcgtgtttttaacagatctcggcctaatacgcctccttccagaggtatggagtctatttctgtaggaaacattcctacggaaaaggtgttgccggaagaacaattgcgaaaaaagtcattggaaactattaaagaattctacagtgccaaagacgagatggaagttgccctgtgtgtcaaagatttgaatgctcccagcttttatccatcaatgatatccatctggataactgattccttcgagaggaaggacatggagaggggtctacttgctaagcttctggtcaaccttgcaaaatctcgggacaccttgttaagtcgacctcaatttgtagaagggtttgaatctgttctagcttgtttggaggatgctgtaactgatgctcctaaagcaccagaatttcttggtggtatctttgctaaatttgtgctggaaaatgtgcttcccatggcagaaattggaaggttaatttatgaaggtggggaacaacaaggacagcttgtagaaatagggcttgcagctgaagttcttggaagcgtcctggagattattaaaactgaaaaaggagaacaggtcttgaaagaaatttgcacgggctctagtttacgtctagaaaactttcggcctccgaactctaaaagggcattgacgctagataaatttatctaa

SEQ ID NO: 9 - protein, 1775 aa, Apium graveolens
MSVNQSRAEKSESGQHRKTNRSGNSSGGQRNFPGGGGKGSGAATAPTPSLSTSRSFKKPSNAQGSQIRVVNSDIRNDATAGGNVAVSGSARGVQNGAHSQSHGGSDAPAVVKQNVTPTPKINRGVPKAPAPNNTPVSSNTMAPSTPVKGGGFPLQFGSISPGLMQVPARTSSAPPNLDEQKREQARYESSRVMPVLPAQSVPKQNVPTNNSATSDQPNIVDTHSMSKARRDVQVAPGPHAIQTQKPSVHPISGMPMQMPFHQQHIPVQFGNPNPQLQSQSMVNSSMPMPMGMPFPMGNPSQVQQQLYIQGLPPHMMPPQGVMHQGQGVNFSSQMGTQLPHMGNMGMNINPQFSQQQPGNFGGARKTVKITHPDTHEELSLSKRTDTYKESGSSAPRSHTNIPPQSQPISSFPPGRPSNFYPNSYSQGSVFYTGPNSLHLSSNQVPPNSQAPRLYKQVTVKPAASPRLEKIAETSLPASSPTVEKNIPKISGQEGEATSIRTERDPEKTSDMSSQKSEYTSIPVQIKGTGGVSDSASASSPLPSSIPCTPPENSEYSTGSSITVVAKNDLLDRSNDNDEKHSKKSQSVLQNQIDRPVAPSLPEVVEDTRSSSIPEAQPGEENVRESETSVAVDLNTNSSEVQGIHELEKSDGVDSAEMSADVSVDNKPKHEAMRTQESRNILLSEPNADAAGCVASAKSDSPRPYEHADQTESLIMEVKDKQLKLEMEPVHLTKGDADADLSTSIVGTVDCLITETSSLSLGSTISHDYDKTSASDASTIIDNTMASTETESNKLDQKSQILSVPSQSEPPCETEIADSDSTELLSSSNDLKDKPVPETIMSKNTNTKKKRREALQKADRAGRIADLYMAYKGPDEKKENPIDVESSETSSSQAKELASGFSPKDVPNESKAEPDDWEDAADLSTPKLDDGKLLGEVKDHIEDKTLMDRKYSRDFLLKFSEQCKDLPVGFKITLDIAEALVLSIGNVPLPSPGRNDDRSMGRSPSGRRGVGMGMGDDDKWSKVPGPLPPGLDMGYGSHANNVFQPGANFGVLRNPRVQSPVMHSSGILSGPVHSMGPQYGMQRTNSDADKWQRATNFQRGLMPSPRTPAQVMHKAERKYEVGKITDEEQAKQRQLKAILNKLTPQNFERLFEQVKQVNIDNAGTLTGVISQIFDKALMEPTFCEMYADFCYHLAGDLPDFNEDNEKITFKRLLLNKCQEEFERGEREQEEANRAEEEGEVKQSDEEREEKRVQARRRMLGNIRLIGELYKKKMLTERIMHECIKKLLGQYQNPDEEDVEALCKLMSTIGEMIDHPKAKEHMDAYFDMMAKLSNNMKLSSRVRFMLKDSMDLRKNKWQQRRKVEGPKKIEEVHRDAANERQAQANRLARGPSMGSSFRRGQQPMDFAPRGSNVLSSPNSHMGGFRGAPQQPRGYATQDIRTDERHPFDNRNLLPQRPLGDSITLVPQGGLARGMSIRGQPPMLSVPFSDMHNQDSRRTTPGLNGYGSVTDRPVYTSREELIPRSAQRFVSPSAHDHMNITDGNSTYMNREMPNPDRVFNRSRPNTPPSRGMESISVGNIPTEKVLPEEQLRKKSLETIKEFYSAKDEMEVALCVKDLNAPSFYPSMISIWITDSFERKDMERGLLAKLLVNLAKSRDTLLSRPQFVEGFESVLACLEDAVTDAPKAPEFLGGIFAKFVLENVLPMAEIGRLIYEGGEQQGQLVEIGLAAEVLGSVLEIIKTEKGEQVLKEICTGSSLRLENFRPPNSKRALTLDKFI

SEQ ID NO: 10 - DNA, 10368 bp, Apium graveolens
  source 1..10368: mol_type = genomic DNA
caattataaatgttcagaatatttgttgataaactgaaatttttggatgtttattaaaaaccatggatgtttgggattctggataatatatctatataaaaaatatgaagagggctatattctataaaacccaacccaagtgaccatctagagataaatatatctgcaatatgaagagattcactgatagggctggacataaaatttcttctatgcagattacaataatcatggctacacttagataaatttgatctagcgtcaatgcccttttagagttcggaggccgaaagttttctagatgtaaactagagcccgtgcaaatttctttcaagacctgttctcctttttcagttttaataatctccaggacgcttccagaacttcagctgcaagccctatttctacaagctgtccttgttgttccccaccttcataaattaaccttccaatttctgccatgggaagcacattttccagcacaaatttagcaaagataccaccaagaatttcggtgctttaggagcatcagttacagcatcctccaaacaagctagaacagattcaaacctggcaaaaaaacaattatgaagtgaaaagaacaccagacaacatattttgaccttctaggataaaacttaagtcagtgaagtggtgtaagcaagaatcaggtaaccattacatacccttctagcaaattgaggttcgacttaacaaggtgtttcgagattttgcaaggttgaccagaagcttagcaagtagacccctctccatgctccttcctctcgaaggaatcagttatccagatggatatcattgatggataaaagctgggagcattcaaatctttgacacacagggcaacttccatctcgtctttggcactgagatttagagcatcttttgagtcgaatatacagtaaaactgaaagaggaagcagggctgtatctcaaaatactttaatcaactcatcacaactttcgcaaacaaagcaaatgcctgttattctagatgaagaatcctatatgaaagaacctgggctacggctgcgtaacaagccagatcaaatagtcaaaagagcctgggagagggcctagtaacaagccggatcaaatattgacacttgcccaggctgacagagtttcaggagtacaaggcctaaatcggactaatactacggccatggctgaaagcaatgattaactagaaagggacctcaatctgaatagcaaatggagaagtaagccaatcctaaatagaaacatgctccttaattttgaagggagaattcaaaacctagcaagttcaaaatttatcatgtacaccaatctcattaggcttattgtacaggaaatttcgtttgactaattcccaccactcaaagaagggaacagccagccatcaatgaattgactaataataagtggtaacaatagatggactcatagagtactaggaatacaagacaacaaagacctaagaccattttaagctgtacgcaaacagtgatgcaagaaaaagcctctcctatgtgtgcatataagttgatgggtaactacaattttgaaaagatggactctcacgaaggtatagtggaaggaagcaacatagaatataatattatactgcatttcttcacagtagcaatcattatgtatactggctcaccttatttacaataatcattttacttttcctgtattttcataccaaaattcattgataactccaaaaatgaactatttgactacatagatatgacaacctatgttacccggactcgggtatgggtgtcggacacgggttcatatccaagtgtcggactcggcaatattatgaaaaatttacatgttttttgcctaaaataagtgtccaagtgtctatacccatgtccgagtgtcgagtgtccgacacgggtactcgaagcaaaatgaagagtccgggtaactaagatgacaactgaggctctactaaaggcctagaaatcagctataaacagtatttaggtatatttcgaattacacagggttgactttaaaattaatgtttattttgtaaaatatataaatgttagctctctctcgacttgctagtatctttaatccttctaaactataaccgtcaattagtaaaggtgcatcaagattcaaaatactacacctagaaacacaagggtaaacacaaaattctgaagattcacattctttgactgtttttattgatagcaatagccgatgatctgctgggcgcctaatgacagcaagataaaatgcaaatacactaaaaagagcagatataaagtacacagcttcatattgttgcaagaccaccgagagtcaaaatacacattcaccccatcaaatctcagacttaaaaaaacaatgaacttgtaaaactagcatatatgatatgttaatttctatcgtacacaacacacggagtaaaagggcaattaaaattagctttttagttgaaaatataattaaaaaatcagccacaagcaagcaacaagttatcatcatacctgtagaattctttaatagtttccaatgacttttttcgcaattgttcttccggcaacaccttttccgtagaaatgtttcctacagaaatagactccatacctctggaaggaggcgtattaggccgagatctgttaaaatacgatctggattctgcatttccctgttcatataagtcgaatttccatccgttatattcatgtggtcatgagcagacgggctgacaaatctctgagcagatcgcggaatcagttcctctcttgaggtataaactggccggtctgttacagaaccgtacccattgagaccaggggtagtccttctggaatcttgattgtgcatgtcagagaagggtacgcttaacattgggggctgtcctctaatagacattcccctggcaagaccaccttggggcacaagggtaatggagtcaccaaggggtcttctgtggcaatagagaattcctgttatcaaaagggtgtctctcactgtcctaactatcttgagtggcataaccgcgtggttgttgtggtgctccacggaatcctcccatatgggaatttggcgatgataatacatttgaccctcttggagcaaagtccatgggttgttgaccccttctaaaagatgaacccatactcggtccacgagctgaatctgttagcttgtgcctgcctctcattcgcagcatccctgtgcacttcctcgatcttttttggaccttcaactttcctcctctgctgccacttgtttttcctgcaggtccattgaatccttcagcatgaacctcactctcgaagagagcttcatgttattagatagcttggccatcatatcaaaatatgcatccatgtgttccttggctttagggtgatctatcatctctcctattgtgctcattagtttgcacaaggcttctacatcttcctcatcaggattttgatactggcccaatagtttcttaatgcattcatgcatgatcctctctgttaacatctttttcttgcataattccccaataagtcttgatgttgcccaacattcgccttcgtgcctggactctcttccttccctttcttcatcagattgtttaacctcaccttcctcttcagctctgttggcttcttcttgttccctctcacctctctcaaactcctcctggcacttattcagaaagtagcctcttaaatgtaattttttcgttgtcttcatcaaaatcaggcaaatcgccagcaagatgatagcagaaatcagcatacatctcacaaaatgtcggttccataagagctttatcaaagatttgagatatcacaccagtgagagtaccagcattgtcaatgttaacttgtcttgacttgctcaaatagtctttcaaagttttgcggtgttaacttattcagaattgctttcaactgcctttgctttagcctgttcctcgtcagtcattttacccacttcatactttctctagccttatgcatcacctgtgcgggagtccgaggagaaggcattagacctctctgaaaattagtcgcccgctgccatttgtcggcatcagagtcagcttcgctgcatttctatactgaggacccatagaatgaactggcccagataggattccaccggaatacataactggtgattgaacacgtggatttctcaatactccaaaatttgctccaggtcgaaaaacattgtttgcatgacttccataacccatatccagtacaggggaagaggcccgggtaccttactccatttatcatcatcgcccatacccatgccaacaccacggcgacctgatccagaccttcccattgacctatcgattatctcttcctgggactgggcaaggaacattgccagtagacaatacccaatgcttctgcaatatctggggtgatctcaaagccctcaggaagatccttgcattgctctgaaaatttaagaaggaaatctctagagtactttctgtccattaaagtttttgtcctcaatgtgatcttttacctctccaaggagcttcccatcatcaagttttggagttgataaatctgcagcatcttcccaagtcatctggttcagctttactctcatttggaacatctttcggagaaatttagaagccaactctttagcttggctagaagatgtttctgagctctcaacatcgatcgtgattttctttcttttcatctggacctttatatgccatataaagatcagcagttgtaccagcacggtttgccttttgtaaagcttcttttctcttcttctttgtatttgtattctttgacataattgtctccggcacaggtttatctttcaaatcaattgatgaagaaagcaattctgtgctatcactatcagcaatttcagtttcacaaggcggctcagactgagatggaactgaaagaatttgtgatttctgatctaacttgtcggattctgtttctgtactagccatggtattatcgattatagtagaagcatccgaagctgaagtcttatcataatcatgtgagattgtggatccaagagacaatgaggatgtttcagtaatcaggcagtccactgtcccaactatagaggttgataaatcagcatctgcttcacctttttgtgaggtgtacaggttccatctccaacttcagctgtttatctttcacttccatgataaggctctcagtttgatcagcatgttcatatggtcttggtgatatcggattttgcacttgccacacaaccagcagcatctgcatttggctcagagagcagtatgtttctactttcttgagttcgcatagcttcaatgttttggcttattatccacagatacatcagctgacatttcagcagaatgactccatcgcttttttccaactcatgaattccctgtacctcagaactatttgtatttaagtcaacggcaacagaggtttctgattccctgacattttcctctcctggctgagcttctggtatagaagatgatcttctgtcctctacaacttcgggtaacgaaggggcaacaggtcgatcaatctgagaacaaaatttgttaaaaccatacactacagcaactgaagtatactgctggaataaaaatcttaaaagagggtaatacctgattctgcagtacagataggctctcttactgtgtttctcatcattatcattagacctatccaatagatcattttttgcacctacagtaatactagagcctgtggaatactcggaattttcaggaggtgtactaggaatagaacttggtagaggggctagatgcggaagatgaatcagatactccgccagttgctttaatttgaacaggtatcgatgtatactctgacttctgtgaggacatatcacttgtctttccaggatctctttcagtacaaatagatgtagcttctcctctaccatatacttgaggaatatttttctcaacagtaggtgagcttgctggcaaagaagtttcagcaattttttccaagcgtgggagctgctggcttcacggtgacctgtattttaaatgagcatgtaccgtaaataacatttcagtaatggcatgcaacaaaataccagcagtggataaaacaacacggtaatacggtttaataaagacaggctaacctgtttatataaccttggagcctgtgaatttggaggaacttggttactgcttaaatgaagagaattaggacctgtataaaatacggaaccctgactgtaagaattagggtaaaagttacttggacggccagtaggaaaagatgaaataggttgggactgaggtggtatattggatgtgatcgcggagcagatcgaaccagattctttgtaagtatcagtctttttagataaactcaactcttcatgagtatcagggtgagttatcttaactgttttacgagcaccaccaaaatttcctggctgctgctgcgagaactggggattgagtgttcatccccatattccccatgtgaggtaactgggtacccatttgcgaggagaaattgacaccctgactctgatgcatgacaccctgaggtggcatcatgtggggtggaagaccctgaatatacagttgctgctgcacttgagaaggatttcccatagggaagggcattcccattggcattggcatagagctatttaccatgctttgggactgcaactgtggattaggattcccaaattgaactggtatatgttgctggtgaaatggcatttgcataggcatcccagaaatagggtgaacagaaggcttctgagtttggattgcatgtggaccaaggtgcaacttgcacatctcttgcttttgacatcgaatgagtgtcaacaatattaggttggtcagaagttgcagaattattggtaggcacattctgcttaggaacggattgggctggcagcacaggcataactcttgatgattcatagcgagcctgcaaaaataattgacaagcaggaaaggaagttgtcaaacaattaatatcagcagggataaagagcagagcataatcagcctattctgtaatttaatcaactcatctcattgcactagggccaaaagtcattctacagaaagcacatcatattacaattaaccaaaccctttttcaagctctataatagattactcaaaaactgagattgcaatttcaagtgtgtcgttcgaaaaaatagagatgcaacaaatgtgggacaccacaaaatgtagcctatccatttggaccagaagggggatcataaagtaatatacttcacatcaaccatttagaaatcaaagttacaaatgatctagcctccctattataataaagagaatactgatattaaaaatattcatctagaaattgcaagtaaatcctatttgcaagcccacccccacatcacaatgaacaaaaaacatggcaacaatgtttaaaacctgttcccgtttctggctcatccaaatttggcggcgctgagctagtttcgagcaggtacctaaaccacaatccgaaatcattccagtaattataattatatgaagaccaatatcattcttaagcttcaggtaactgagatagctaacaatatacattcttgatgaaaactttcagccctacctgcatcagactaggactctattgatcaaacctggagaggaaaacctccacctgagccacgaccacaaaactccaattagcttattatgaatacagaagttcatatatttaacttcatcatgtacaatgccaaatatttacatagaacgacacttaacctttagcaggggttgaaggggccattgtatttgagcttacggggtattatttggagcaggagctttcggcatatccccgattaatttttggtgccggagtaacattctgcttgacaacagctggagcatcagaccctcctacaacaaggacaacaaaataaaaaccatactcgaccatcacaccacattcaaaaacaaaaacaattaaaatccttgaacaaaatctctttcaataccatgtgattgtgaatgagcgccattcttgcacaccacgggctcaaccagaaacagcaacattaccaccggcagtagcatcattcctaatatccgaattcacaacccgaatttgcgatccttgcgcattactaggcttcttaaaactacaaaaaataaaaaaaaattattagaatcatcagaaaagtacacaaaacagacaagtatactctataacttcatactgtccggaccaaagattctattacttaattaataaagcttacaaattaactacaggtaaaacaaacctaagtatccttgataaattaaaaaaaacacattctttaatcttagtatataaccaataatcacgtatctaatttatccaatcaagcactaagacaatcttaggtcacgtcaatcacccccacatattaatttatgaagactaaaaaccaatcaagcacttattagctaactaatcaatcacgtagtatttaattatcgagattatgtaaccaaccaagtaccaacaatcttaggttactaaccaatagcgtattatattgtcaagattactaaccgagtaccaacaatcttaggtcactaaccaattgcgtatctaattattcaaattactaaccaacccagtaccaattaatcttagacaactaacaaattgcataataatttatcgaggtctaaatgtacacacaattacaaaaaacaaagacaggaaacccaatataccttcgactagtcgaaagcgaaggtagaggggcggtggcagcaccgctacccttaccaccaccgcctgaaaaattcctctgcccaccggagctatcacccggatcgattagtttttcgatactgtcccgactcgctcttctgagccctagattgattgacggacataaatccaaaccctaatcacctccaatcttaatcaaatcaacaattttctgcaccaaaaacacagatctaattcattaatcacataatcaacaaaaattcaaaacacattacaatcaattcatacacacaaacatatatacatgtaattaaagatgcttaattgattaaagattgaatctttttggtgaaattgagtgaatagagtgaagatatatgtataataattgtgtaggcgtgtaagaattgtgtgtatatgtgtatgtatagattggattgaattggatggaagggggtgaagaagaggaagacagagaaggaaaaactgaacccaagtgtatgtgtgtgtttgtgtgaattgtgtgtgtgagaaaagaagtgtgttttaacttttatatgtgtatatataaaatcaagaattaaagtatcattgttttcaaattggaattcagaaaaaattaaattaaaataatataattgttgtttaaaagcttccttttgagaaaattacaaattttagattatatatggacgatggtcaaaaatattattttttaatataatatgacaaacatactatttgtaaaattgatggtcaaaaatatcaaccaagtacatatttcgaagaattgataatctaatacgtatttgatgagctacggagttttacccgcgatacgcaagattgttatccgaaatttgttaaacgtaaattgttatataaatgaaaaaaaatcggtatatcgtattccgagatgatgaactcttaattatattaacgaacaaataaaaatacataaagatgcaccaaaaaatataaatcgtcactttcaaagaaaaatacaggaacctattgtttgattaaaatctcacatcaatgtcaggattatattcgccacaaagatttcagcgttaagcaattgacgattataagaagtgaacatagtaacaatctgaatgctttgttaattaatataacgcgtacattacttaacggaaacgtaaatgtgtaccagtgtggccagatagactattcaccttcttataattacactttactacattagtcttttgcatgacactatttttcttgggagatgcaatatataaaatgctttaactctaatgtccccacaaatgtcatgtcgtgtttaacaatacatgtttgtttaattgtacaaacagttaaatctcgatattataatactttataacttctgaaatatgggttaattatatctaacttcggtgaatctaaaattaagtaatatggtttaatatcatatatgttagcataaaatatgttagtataaaatataaagtttgtcatacaacgataacattttcttttaatttaatattagtaacaaattatccctttaatatcattctaagcaataatataaacaaactaatgataactgatcttctatataatt

SEQ ID NO: 11 - DNA, 3737 bp, Apium graveolens
  source 1..3737: mol_type = other DNA
atgtatatatggctcagaagagcgagtcgggacagtatcgaaaaactaatcgatccgggtgatagctccggtgggcagaggaatttttcaggcggtggtggtaagggtagcggtgctgccaccgcccctctaccttcgctttcgactagtcgaagttttaagaagcctagtaatgcgcaaggatcgcaaattcgggttgtgaattcggatattaggaatgatgctactgccggtggtaatgttgctgtttctggagggtctgatgctccagctgttgtcaagcagaatgtggaggttttcctctccaggtacctgctcgaaactagctcagcgccgccaaatttggatgagccagaaacgggaacagagatgtgcaagttgcaccttggtccacatgcaatccaaactcagaagccttctgttcaccctatttctgggatgcctatgcaaatgccatttcaccagcaacatataccagttcaatttgggaatcctaatccacagttgcagtcccaaagcatggtaaatagctctatgccaatgccaatgggaatgcccttccctatgggaaatccttctcaagtgcagcagcaactgtatattcagggtcttccaccccacatgatgccacctcagggtgtcatgcatcagagtcagggtttctcgcagcagcagccaggaaattttggtggtgctcgtaaaacagttaagataactcaccctgatactcatgaagagttgagtttatctaaaaagactgatacttacaaagaatctggttcgatctgctccgcgatcacatccaatataccacctcagtcccaacctatttcatcttttcctactggccgtccaagtcaccgtgaagccagcagctcccacgcttggaaaaaattgctgaaacttctttgccagcaagctcacctactgttgagaaaaatattcctcaagtatatggtagaggagaagctacatctatttgtactgaaagagatcctggaaagacaagtgatatgtcctcacagaagtcagagtatacatcgatacctgttcaaattaaagcaactggcggagtatctgattcatcttccgcatctagcccctctaccaagttctattcctagtacacctcctgaaaattccgagtattccacaggctctagtattactgtaggtgcaaaaaatgatctattggataggtctaatgataatgatgagaaacacagtaagagagcctatctgtactgcagaatcaggtattaccctcttttaagatttttattccagcagtatacttcagttgctgtagtgtatggttttaacaaattttgttctcagattgatcgacctgttgccccttcgttacccgaagttgtagaggacagaagatcatcttctataccagaagctcagccaggagaggaaaatgtcagggaatcagaaacctctgttgccgttgacttaaatacaaatagttctgaggtacagggaattcatgagttggaaaaaagcgatggagtcattctgctgaaatgtcagctgatgtatctgtggataataagccaaaacattgaagctatgcgaactcaagaaagtagaaacatactgctctctgagccaaatgcagatgctgctggtgaagcagatgctgatttatcaacctctatagttgggacagtggactgcctgattactgaaacatcctcattgtctcttggatccacaatctcacatgattatgataagacttcagcttcggatgcttctactataatcgataataccatggctagtacagaaacagaatccgacaagttagatcagaaatcacaaattctttcagttccatctcagtctgagccgccttgtgaaactgaaattgctgatagtgatagcacagaattgctttcttcatcaattgatttgaaagataaacctgtgccggagacaattatgtcaaagaatacaaatacaaagaagaagagaaaagaagctttacaaaaggcaaaccgtgctgaaacatcttctagccaagctaaagagttggcttctaaatttctccgaaagatgttccaaatgagagtaaagctgaaccagatgacttgggaagatgctgcagatttatcaactccaaaacttgatgatgggaagctccttggagagtcccaggaagagataatcgataggtcaatgggaaggtctggatcaggtcgccgtggtgttggcatgggtatgggcgatgatgataaatggagtaagcgggcgactaattttcagagaggtctaatgccttctcctcggactcccgcacaggtgatgcataaggctagagaaaactatttgagcaagtcaagacaagttaacattgacaatgctggtactctcactggtgtgatatctcaaatctttgataaagctcttatggaaccgacattttgtgagataggctactttctgaataagtgccaggaggagtttgagagaggtgagagggaacaagaagaagccaacagagctgaagaggaaggtgaggttaaacaatctgatgaagaaagggaaggaagagagtccaggcacgaaggcgaatgttgggcaacatcaagacttattggggaattatgcaagaaaaagatgttaacagagaggatcatgcatgaatgcattaagaaactattgggccagtatcaaaatcctgatgaggaagatgtagaagccttgtgcaaactaatgagcacaataggagagatgatagatcaccctaaagccaaggaacacatggatgcatattttgatatgatggccaagctatctaataacatgaagctctcttcgagagtgaggttcatgctgaaggattcaatggacctgcaggaaaaacaagtggcagcagaggaggaaagggtcaacaacccatggactttgctccaagagggtcaaatgtattatcatcgccaaattcccatatgggaggattccgtggagcaccacaacaaccacgcggttatgccactcaagatagttaggacagtgagagacacccttttgataacaggaattctctattgccacagaagaccccttggtgactccattacccttgtgccccaaggtggtcttgccaggggaatgtctattagaggacagcccccaatgttaagcgtacccttctctgacatgcacaatcaagattccagaaggactacccctggtctcaatgggtacggttctgtaacagaccggccagtttatacctcaagagaggaactgattccgcgatctgctcagagatttgtcagcccgtctgctcatgaccacatgaatataacggatggaaattcgacttatatgaacagggaaatgcagaatccagatcgtattttaacagatctcggcctaatacgcctccttccagagccctgcttcctctttcagttttactgtatattcgactcaaaagatgctctaaatctcagtgccaaagacgagatggaagttgccctgtgtgtcaaagatttgaatgctcccagcttttatccatcaatgatatccatctggataactgattccttcgagaggaaggagcatggagaggggtttgaatctgttctagcttgtttggaggatgctgtaactgatgctcctaaagcaccgaaattcttggtgcttgtagaaatagggcttgcagctgaagttctggaagcgtcctggagattattaaaactgaaaaaggagaacag

SEQ ID NO: 12 - protein, 1245 aa, Apium graveolens
MYIWLRRASRDSIEKLIDPGDSSGGQRNFSGGGGKGSGAATAPLPSLSTSRSFKKPSNAQGSQIRVVNSDIRNDATAGGNVAVSGGSDAPAVVKQNVEVFLSRYLLETSSAPPNLDEPETGTEMCKLHLGPHAIQTQKPSVHPISGMPMQMPFHQQHIPVQFGNPNPQLQSQSMVNSSMPMPMGMPFPMGNPSQVQQQLYIQGLPPHMMPPQGVMHQSQGFSQQQPGNFGGARKTVKITHPDTHEELSLSKKTDTYKESGSICSAITSNIPPQSQPISSFPTGRPSHREASSSHAWKKLLKLLCQQAHLLLRKIFLKYMVEEKLHLFVLKEILERQVICPHRSQSIHRYLFKLKQLAEYLIHLPHLAPLPSSIPSTPPENSEYSTGSSITVGAKNDLLDRSNDNDEKHSKRAYLYCRIRYYPLLRFLFQQYTSVAVVYGFNKFCSQIDRPVAPSLPEVVEDRRSSSIPEAQPGEENVRESETSVAVDLNTNSSEVQGIHELEKSDGVILLKCQLMYLWIISQNIEAMRTQESRNILLSEPNADAAGEADADLSTSIVGTVDCLITETSSLSLGSTISHDYDKTSASDASTIIDNTMASTETESDKLDQKSQILSVPSQSEPPCETEIADSDSTELLSSSIDLKDKPVPETIMSKNTNTKKKRKEALQKANRAETSSSQAKELASKFLRKMFQMRVKLNQMTWEDAADLSTPKLDDGKLLGESQEEIIDRSMGRSGSGRRGVGMGMGDDDKWSKRATNFQRGLMPSPRTPAQVMHKARENYLSKSRQVNIDNAGTLTGVISQIFDKALMEPTFCEIGYFLNKCQEEFERGEREQEEANRAEEEGEVKQSDEEREGRESRHEGECWATSRLIGELCKKKMLTERIMHECIKKLLGQYQNPDEEDVEALCKLMSTIGEMIDHPKAKEHMDAYFDMMAKLSNNMKLSSRVRFMLKDSMDLQEKQVAAEEERVNNPWTLLQEGQMYYHRQIPIWEDSVEHHNNHAVMPLKIVRTVRDTLLITGILYCHRRPLGDSITLVPQGGLARGMSIRGQPPMLSVPFSDMHNQDSRRTTPGLNGYGSVTDRPVYTSREELIPRSAQRFVSPSAHDHMNITDGNSTYMNREMQNPDRILTDLGLIRLLPEPCFLFQFYCIFDSKDALNLSAKDEMEVALCVKDLNAPSFYPSMISIWITDSFERKEHGEGFESVLACLEDAVTDAPKAPKFLVLVEIGLAAEVLEASWRLLKLKKEN

SEQ ID NO: 13 - DNA, 160 bp, synthetic construct
  source 1..160: mol_type = other DNA
tcaatttggacatgtttcttcttaataataattttaataataataatttcaaaaatgttcagaacctgtttgggaactaggaattgacaaccgattgattttccggaagttctacctcaggatcatgagtcgctgctttctcgcaggaagcatgtgctgg

SEQ ID NO: 14 - DNA, 462 bp, synthetic construct
  source 1..462: mol_type = other DNA
ggtaatgttgctgtttctgaatccttctcaagtgcagcaactcatgaagagttgagttaaaatattcctcaagtatatcaagtgatatgtcctcacagcacctcctgaaaattccgagtattccatttaacaaattttgttctcagattgatcgacctgttgccccttcgttacccgaagttgtaccaaatgcagatgctgctggtgaagcagatgctgatttaaagaatacaaatacaaagaagaagagaaaagaagtcaactccaaaacttgatgttgagagaggtgagagggagaagaggaaggtgaggttaaaagaaagggaaggaagagaaagaaaaagatgttaacagatgaatgcattaagaaactattgggccagtttgctccaagagggtcaactgtaacagaccggccagttcagttttactgtatattcggaagttgccctgtgtgtcaaa

SEQ ID NO: 15 - DNA, 24 bp, synthetic construct
  source 1..24: mol_type = other DNA
ttttccaagcgtggggaagctgct

SEQ ID NO: 16 - DNA, 98 bp, Apium graveolens
  source 1..98: mol_type = genomic DNA
taaaagatgttaacatgtgccttcaagtctctttctgcattcttgatggtwttagcatttttattaacttcttttttgtgtgtgttacttaatatgta

SEQ ID NO: 17 - DNA, 101 bp, Apium graveolens
  source 1..101: mol_type = genomic DNA
aaaaagttccaaaacaatcaaacataaaatagattcaacctaccattactwaaaatrtttatgttctatgtcataaagcctcaatattctgaaattacaca

SEQ ID NO: 18 - DNA, 101 bp, Apium graveolens
  source 1..101: mol_type = genomic DNA
gctgcaaattttccccatggccgccgccttacgcctctatatctcttccastcttgtggagactgactttcaggtgccacaacctctatttttatgtcctc

(19) United States
(12) Patent Application Publication
(10) Pub. No.: US 2026/0286379 A1
(43) Pub. Date: Sep. 24, 2026

(54) CeMV Resistance in Celery

(71) Applicant: Bejo Zaden B.V., Warmenhuizen (NL)

(72) Inventors: Jacob van Dorp, Warmenhuizen (NL); Adriana Dorien Haarsma, Warmenhuizen (NL); Babette Rens van den Berg, Warmenhuizen (NL); Klaas Schotanus, Warmenhuizen (NL); Timo Christiaan Petter, Warmenhuizen (NL); Albertus Johannes Maria Schrijver, Warmenhuizen (NL)

(21) Appl. No.: 19/164,250

(22) PCT Filed: Mar. 11, 2024
(86) PCT No.: PCT/EP2024/056373

§ 371 Date: Sep. 11, 2025

(30) Foreign Application Priority Data
Mar. 13, 2023 (WO) PCT/EP2023/056353
Aug. 15, 2023 (WO) PCT/EP2023/072433

(51) Int. Cl. C12N 15/82; C12Q 1/6895

(52) U.S. Cl. CPC C12N 15/8283; C12Q 1/6895; C12Q 2600/13; C12Q 2600/156

(57) ABSTRACT

Provided herein are CeMV resistant celery plants, methods for identifying CeMV resistant celery plants, methods for producing CeMV resistant celery plants and means for producing or identifying CeMV resistant celery plants. Specifically, the present invention relates to CeMV resistant celery plants including a genomic region between 80,545 and 80,589 cM on chromosome 10, wherein said genomic region includes one or more mutations causing said CeMV resistance.

### CROSS-REFERENCE TO RELATED APPLICATIONS

This application is the United States national phase of International Patent Application No. PCT/EP2024/056373 filed Mar. 11, 2024, and claims priority to International Patent Application No. PCT/EP2023/056353 filed Mar. 13, 2023, and International Patent Application No. PCT/EP2023/072433 filed Aug. 15, 2023, the disclosures of which are hereby incorporated by reference in their entireties.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2504400.xml. The size of the file is 49,519 bytes, and the file was created on Aug. 20, 2025.

### BACKGROUND OF THE INVENTION

### Field of the Invention

The present invention relates to CeMV resistant celery plants, methods for identifying CeMV resistant celery plants, methods for producing CeMV resistant celery plants and means for producing or identifying CeMV resistant celery plants.

Celery (Apium graveolens) is a member of the Apiaceae family, a family of aromatic flowering plants that includes carrot, parsnip, parsley, coriander, fennel and dill. Three types of celery, which are morphologically distinct, are generally cultivated. Celery or stalk celery (A. graveolens var. dulce) is grown for its long solid petioles (leaf stalks), celeriac (A. graveolens var. rapaceum) for its thickened stem (hypocotyl), and leaf celery (A. graveolens var. secalinum), which has thin stalks, for its leaves.

Celery is a popular vegetable and is consumed around the world. In North America and Europe, the crisp petiole (leaf stalk) of celery is usually eaten raw or used as an ingredient in salads, juices and soups. In Europe, the thickened hypocotyl of celeriac is shredded and used in salads. Celery leaves have a strong flavor and are used to season soups or stews, or used as a dried herb. Celery seeds contain various substances with potentially health-promoting properties and are, therefore, used in dietary supplements and alternative medicine. The seeds can also be crushed and mixed with salt to produce celery salt for the seasoning of food, although in some cases, celery salt is also made from an extract of the roots or using dried leaves.

Celery has a long history of cultivation. First described by Linnaeus in Species Plantarum in 1753, celery is presumably native to the salt marshes of Europe and the Mediterranean but is currently grown in many regions around the world, including Australia, South Africa and South America. The largest producer of stalk celery is the USA, especially the state of California, followed by Mexico. Celeriac is predominantly grown in Europe, and Chinese or leaf celery is the most common type of celery grown in Asia.

Celery grows best in temperate climates with mild or cool weather. Stalk celery is grown from seeds sown in a hot bed or the open garden depending on the season. When the plants reach a height of 15 to 20 cm, they are transplanted to deep trenches, which are gradually filled up with soil to limit exposure to the sun. This process is called blanching and yields plants that are less bitter and have a mild, sweet and aromatic taste. Several modern cultivars will blanch spontaneously without this treatment and are called self-blanching. Due to the very high uniformity of modern cultivars, fields are only harvested once, roughly three months after planting. After removal of the leaves and stalks, celery can be stored for several weeks at temperatures between 0 to 2° C.

Like most crops, various pathogens challenge the cultivation of celery and celeriac. Examples are the ascomycete fungus Septoria apiicola which causes celery leaf spot or late blight; the ascomycete fungus Fusarium oxysporum causing Fusarium yellows, also known as Fusarium blight or Fusarium wilt; and the fungus Cercospora apii, the cause of early blight. Other pathogens are known such as viruses and several insects, like leaf miners and shield bugs (Graphosoma sp.).

Celery mosaic virus (CeMV), a commercially important pathogen of celery, is a member of the largest plant (positive) RNA-virus family: Potyviridae, genus Potyvirus. Viruses in this genus are generally transmitted by aphids and/or mechanically. The aphid Myzus persicae (Sulz.) is a known vector of CeMV. Next to celery, CeMV is known to infect parsley, carrot, coriander, parsnip and dill. Infected plants show the following symptoms: narrowing of the leaves, mosaic and/or mottling patterns on leaves, vein clearing, yellowing of foliage and distorted foliage. Plants may appear dwarfed or exhibit loss of vigour. Currently, CeMV is present worldwide (e.g., Chile, Poland, Iran) but it is certainly a problem in western states of the USA and in Florida as well as in Australia.

### Description of Related Art

One way of controlling the disease is to introduce a host-free period of 2 to 3 months, during which no celery is grown in the area. However, when other host crops like carrot, parsley and coriander are grown in that area, the virus can be maintained on these crops and, as a result, this approach is not efficient. Such measure of control is rendered even more inefficient due to presence of weeds that can also serve as host for CeMV. An example of such a weed is wild hemlock in the UK.

Considering the above, there is a need in the art for providing genetically encoded resistance to CeMV in celery.

It is an object of the present invention, amongst other objects, to meet the above need in the art.

According to the present invention, the above need is met as outlined in the appended claims.

Specifically, the above need is met by providing CeMV resistant celery plants comprising a genomic region between 80,545 and 80,589 cM on chromosome 10, said genomic region comprises one or more mutations causing said CeMV resistance.

### SUMMARY OF THE INVENTION

According to a preferred embodiment, the present invention relates to CeMV resistant celery plants comprising a mutated genomic region of 8.8 Mbp between base pair positions 80,246,438 and 89,075,890 of the celery genome.

According to an especially preferred embodiment, the present mutated genomic region is derived, originates or is from a celery plant deposited at NCIMB with deposit number 44126 (NCIMB Limited, Wellheads Place, Dyce, Aberdeen, AB21 7 GB, United Kingdom) on the 2 March, 2023.

According to yet another especially preferred embodiment, the present CeMV resistant celery comprises one or more mutations causing the absence of a protein encoded by the cDNA sequence represented by SEQ ID No. 8 in said celery plant.

Preferably, the present one or more mutations are in the genomic region represented by SEQ ID No. 7. The present one or more mutations are deletions, insertions or substitutions in the genomic region represented by SEQ ID No. 7 resulting in the absence of a protein encoded by SEQ ID No. 8.

Also preferably, the present one or more mutations are deletions, insertions or substitutions in the cDNA sequence represented by SEQ ID No. 8 resulting in the absence of a protein encoded by SEQ ID No. 8.

According to an especially preferred embodiment, the present invention relates to CeMV resistant celery plants, wherein the present one or more mutations comprise:
a) deletion of at least 4 consecutive amino acids in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
b) amino acid substitutions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
c) single amino acid deletions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
d) amino acid insertions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus.

According to another especially preferred embodiment, the present invention relates to CeMV resistant celery plants, wherein the present one or more mutations comprise:
a) deletion of at least 4 consecutive amino acids in the amino acid sequence of SEQ ID No. 9 within 20 amino acids of positions 586 and/or 1593; and/or
b) amino acid substitutions in the amino acid sequence of SEQ ID No. 9 within 20 amino acids of positions 586 and/or 1593; and/or
c) single amino acid deletions in the amino acid sequence of SEQ ID No. 9 within 20 amino acids of positions 586 and/or 1593; and/or
d) amino acid insertions in the amino acid sequence of SEQ ID No. 9 within 20 amino acids of positions 586 and/or 1593.

Most preferably, the present CeMV resistant celery plants comprise the genomic sequence of SEQ ID No. 10, or sequences having at least 90% sequence identity with SEQ ID No. 10 or a protein comprising the amino acid sequence of SEQ ID No. 12, or sequences having at least 90% sequence identity with Seq ID No. 12.

According to the present invention, the present genomic region or genomic sequence is homozygously present in the present resistant celery plants.

The present invention also relates to CeMV resistance in celery providing genes, wherein the genes comprises the genomic sequence of SEQ ID No. 10, or sequences having at least 90% sequence identity with SEQ ID No. 10; and to proteins encoded by a nucleotide sequence comprising SEQ ID No. 11, or sequences having at least 90% sequence identity with Seq ID No. 11; and to proteins comprising the amino acid sequence of SEQ ID No. 12, or sequences having at least 90% sequence identity with Seq ID No. 12.

The present CeMV resistant celery plants are preferably stalk celery, celeriac celery or leaf celery.

Considering the beneficial properties of the CeMV celery plants as defined above, the present invention also relates to seeds or plant parts thereof.

The present further relates to method for identifying a CeMV resistant celery plant, the methods comprise the steps of:
isolating genomic DNA from said celery plant; and
establishing the presence of one or more sequences selected from the group
consisting of Seq ID Nos. 1 to 6 or 10 in the genome of said celery plant; or methods for identifying a CeMV resistant celery plant, the methods comprise the steps of:
isolating mRNA from said celery plant; and
establishing the absence of a cDNA represented by SEQ ID No. 8 and/or the presence of SEQ ID No. 11 in said isolated mRNA.

The disclosure of the present invention provided herein allows for methods for providing a CeMV resistant celery plant, the methods comprise the step of introducing, preferably homozygously, a mutated genomic region as defined above into the genome of a celery plant, preferably susceptible, or SEQ ID No. 10.

The present invention also relates to isolated DNA sequences selected from the group consisting of SEQ ID Nos. 1 to 6 and SEQ ID Nos. 10 to 12.

### BRIEF DESCRIPTION OF THE DRAWINGS

The present invention will be further detailed in the examples below. In the examples, reference is made to FIG. 1, wherein

FIG. 1: shows an alignment of the sequence of the functional protein originating from the susceptible plants (SEQ ID NO: 9) with the sequence of the protein originating from the resistant plants (SEQ ID NO: 12). In FIG. 1, (*) represents positions that have a single and fully conserved residue; (:) represents conservation between groups of strongly similar properties with a score greater than 0.5 on the PAM 250 matrix; (.) represents conservation between groups of weakly similar properties with a score less than or equal to 0.5 on the PAM 250 matrix.

FIG. 2: shows an alignment of the sequences of genomic DNA from the susceptible plants (SEQ ID NO: 7) with the sequence of the genomic DNA originating from the resistant plants (SEQ ID NO: 10).

### DESCRIPTION OF THE INVENTION

### Examples

In general, breeding for resistance starts by making a cross between a source of resistance and susceptible genetic material with a high level of agronomical quality. Resistant offspring is selected using DNA markers and repeatedly backcrossed to the agronomically elite parent line. This process ultimately leads to resistant plants with desirable agronomic characteristics.

A celery plant according to the present invention, Apium graveolens 2290733-1850869-2180293, deposit was deposited at NCIMB Limited, Wellheads Place, Dyce, Aberdeen, AB21 7 GB, United Kingdom on the 2 March, 2023 under deposit number NCIMB 44126.

### Example 1. Celery Mosaic Virus (CeMV) Disease Trial

Untreated seeds were sown, germinated, and grown in soil for 4 weeks at a day/night temperature of 20°/10° C. with a 16 h photoperiod (‘day’). After 4 weeks, plants were transplanted in 10×5 cm pots and grown for another 3 weeks at 20°/18° C. At least 20 individual plants of each

CeMV was maintained as lyophilized celery leaves at 4° C. To propagate the virus, susceptible celery plants were mechanically inoculated with CeMV 6 weeks prior to the trial. Subsequently, the viral inoculum was prepared from infected leaves with a concentration of 2 gram of infected leaves in 10 mL Sörenssen buffer (pH 7.0). Per 500 mL inoculum, 15 grams of carborundum was added. The virus suspension was then mechanically inoculated by rubbing the inoculum on the leaves, three strokes per leaf. A total of two inoculations per test plant were performed two days apart.

The trial was kept at day/night temperature of 20°/18° C. with a 16 h photoperiod. Three weeks post inoculation, all leaves were removed from the plants. Six weeks after inoculation, virus symptoms were visible on the susceptible control and the trial was assessed using R (Resistant) or S (Susceptible).

TABLE 1

| Plant | Score |
|---|---|
| NCIMB 44126 | R |
| Golden spartan | S |

The resistant scoring NCIMB 44126 and susceptible scoring Golden spartan were tested for the presence of CeMV after the disease trial by ELISA (enzyme-linked immunosorbent-assay). Leaves of four (including one mock inoculation) plants from each genotype were sampled. The RT-0148 set from Leibniz Institute DSMZ (German Collection of Microorganisms and Cell cultures) was used to detect CeMV by ELISA.

Leibniz Institute, German Collection of Microorganisms and Cell cultures.

The ELISA yielded negative results for NCIMB 44126, indicating the absence of CeMV. The samples of Golden spartan tested positive in the ELISA, indicating the presence of CeMV. All the scores together support the claim that NCIMB 44126 is resistant while Golden spartan is susceptible to CeMV.

### Example 2. Molecular Characterization of Genomically Encoded Resistance to CeMV

An F1S1 population was made by crossing the source of resistance to a susceptible celery line, after which the resulting F1 plant was self-pollinated. At least 2000 seeds were harvested from the F1S1 generation of a cross between the distinctive source of resistance and a susceptible celery line. To perform a QTL mapping, 760 plants of the cross were grown in the glasshouse and tested for CeMV resistance. From each individual plant, leaf material was used for DNA isolation and successive marker analysis. Using SNP markers covering the entire genome, QTLs were found on linkage group LG02. These QTLs are defined by the SNP markers listed in the tables below.

TABLE 2

| | | Position | | | |
|---|---|---|---|---|---|
| SNP | name | (cM) | Res* | Alt* | Sequence |
| 1 | 0674 | LG02_080.545 | T | A | TAAAAGATGTTAACATGTGCCTTCA |
| (SEQ ID | | | | | AGTCTCTTTCTGCATTCTTGATGGT |
| NO: 16) | | | | | [A/T]TTAGCATTTTTATTAACTTC |
| | | | | | TTTTTTGTGTGTGTTACTTAATATG |
| | | | | | TA |
| 2 | 0902 | LG02_080.563 | A | T | AAAAAGTTCCAAAACAATCAAACAT |
| (SEQ ID | | | | | AAAATAGATTCAACCTACCATTACT |
| NO: 17) | | | | | [A/T]AAAATRTTTATGTTCTATGT |
| | | | | | CATAAAGCCTCAATATTCTGAAATT |
| | | | | | ACACA |
| 3 | 0078 | LG02_080.589 | G | C | GCTGCAAATTTTCCCCATGGCCGCC |
| (SEQ ID | | | | | GCCTTACGCCTCTATATCTCTTCCA |
| NO: 18) | | | | | [C/G]TCTTGTGGAGACTGACTTTC |
| | | | | | AGGTGCCACAACCTCTATTTTTATG |
| | | | | | TCCTC |

*(Res) resistant allele; (Alt) alternative allele.

TABLE 3

| | | Position | |
|---|---|---|---|
| Seq ID | | on LG02 | |
| No | SNP | (cM) | Sequence |
| 1 | 1 | 80.545 | TAAAAGATGTTAACATGTGCCTTCA |
| | | | AGTCTCTTTCTGCATTCTTGATGGT |
| | | | [T]TTAGCATTTTTATTAACTTCTT |
| | | | TTTTGTGTGTGTTACTTAATATGTA |
| 2 | 1 | 80.545 | TAAAAGATGTTAACATGTGCCTTCA |
| | | | AGTCTCTTTCTGCATTCTTGATGGT |
| | | | [A]TTAGCATTTTTATTAACTTCTT |
| | | | TTTTGTGTGTGTTACTTAATATGTA |
| 3 | 2 | 80.563 | AAAAAGTTCCAAAACAATCAAACAT |
| | | | AAAATAGATTCAACCTACCATTACT |
| | | | [A]AAAATRTTTATGTTCTATGTCA |
| | | | TAAAGCCTCAATATTCTGAAATTAC |
| | | | ACA |
| 4 | 2 | 80.563 | AAAAAGTTCCAAAACAATCAAACAT |
| | | | AAAATAGATTCAACCTACCATTACT |
| | | | [T]AAAATRTTTATGTTCTATGTCA |
| | | | TAAAGCCTCAATATTCTGAAATTAC |
| | | | ACA |
| 5 | 3 | 80.589 | GCTGCAAATTTTCCCCATGGCCGCC |
| | | | GCCTTACGCCTCTATATCTCTTCCA |
| | | | [G]TCTTGTGGAGACTGACTTTCAG |
| | | | GTGCCACAACCTCTATTTTTATGTC |
| | | | CTC |
| 6 | 3 | 80.589 | GCTGCAAATTTTCCCCATGGCCGCC |
| | | | GCCTTACGCCTCTATATCTCTTCCA |
| | | | [C]TCTTGTGGAGACTGACTTTCAG |
| | | | GTGCCACAACCTCTATTTTTATGTC |
| | | | CTC |

The abbreviations of nucleotides are according to UPAC code:

| Symbol | Nucleotide Base |
|---|---|
| A | Adenine |
| C | Cytosine |
| G | Guanine |
| T | Thymine |
| N | A or C or G or T |
| M | A or C |
| R | A or G |
| W | A or T |
| S | C or G |
| Y | C orT |
| K | G orI |
| V | Not T |
| H | Not G |
| D | Not C |
| B | Not A |

### Example 3. Breeding of Agriculturally Elite Celery Resistant to CeMV

In the first step, a plant being resistant to CeMV (Source) was crossed with a quality line. The offspring of this cross was subsequently (back-)crossed with the quality line (Qline), see Table 4 below. The presence of the CeMV resistance was followed with molecular markers through the backcrossing and also confirmed in the last step.

TABLE 4

| Year | Parent 1 | Parent 2 | Cross id | Harvested | Seedlot |
|---|---|---|---|---|---|
| 2012 | | Source | | S 404 | 1170437 |
| 2013 | Qline B | Source | S 356-1*S 404-9 | T 740 | |
| 2014 | Qline B | Qline B*Source | T 739*T 740-9 | V 626 | |
| 2015 | Qline B | Qline B(2)*Source | V 391-1*V 626-1 | W 887 | |
| 2016 | Qline B | Qline B(3)*Source | W 371-1*W 887-1 | X 8395 | |
| 2017 | Qline A | Qline B(4)*Source | X 369-1*X8395-1 | Y 1052 | |
| 2018 | Qline A | Qline A*Qline B(4)*Source | Y 1052-1*Y | Z 944 | |
| | | | 1053-1 | | |
| 2019 | Qline A | Qline A(2)*Qline B(4)*Source | Z 944*Z 942 | A 1094 | 1900069 |
| 2020 | Qline A | Qline A(3)*Qline B(4)*Source | A 1094*A 1113 | E 729 | 2070413 |
| 2022 | Qline A | Qline A(4)*Qline B(4)*Source | E 729*E 728 | | 2290733 |

### Example 4. Production of F1 Seed Applying CMS

One of the requirements for a modern hybrid variety is that inbreeding, resulting in undesirable off-type plants, is minimized. In celery, a reliable system for hybrid production is available based on cytoplasmic male sterility. Applying this feature for seed production with male and female parent lines, hybrids essentially are resulting 100% from pure cross pollinations.

### Example 5. Gene Providing Resistance to CeMV in Celery

A FASTA file with an Apium graveolens cv. ‘Q2-JN11’ genome has been downloaded from CeleryDB** and subsequently searched for the presence of the markers of Table 3.

The markers map to a single chromosomal location located on chromosome 10, spanning a region of 8.8 Mbp (Chr10: 80246438-89075890) and 41 genes are located within this region. Chromosome numbering and positions in (bp) are is as in CeleryDB**. One of the genes within this region encodes a eukaryotic translation initiation factor 4G-like (eIF4G) protein. Former studies have linked this protein to a role in the 5′cap formation of RNAs.

Previously, it was shown that mutations in the eIF4 complex can lead to resistance against different potyviruses. We hypothesize that a mutation(s) occurred in eIF4G encoding gene of the CeMV resistant A. graveolens and said mutation(s) prevent(s) CeMV from highjacking the translation machinery of A. graveolens, thereby providing resistance.

In the susceptible plants eIF4G has a sequence (Seq ID No 7, for genomic DNA; Seq ID No 8 for cDNA) ref. Table 5. The cDNA sequence (Seq ID No 8) was derived from the annotation as in CeleryDB**. The resistant plants are homozygous and recessive. In the resistant plant the gene encoding for eIF4G (Seq ID No 7, for genomic DNA; Seq ID No 8 for cDNA) comprises at least one mutation, whereby it does not lead to the production of a (fully-) functional eIF4G protein. ** Apium_graveolens_genome_v3 taken from: http://celerydb.bio2db.com/index.html

The mutation or mutations can be of the type: insertion, deletion, frame shift, substitution, mutation, truncation, rearrangement.

Subsequently, a resistant line with resistance gene as in the deposit NCIMB 44126 has been sequenced with Nanopore sequencing. After base calling a de novo genome assembly was created with Flye. The earlier mentioned QTL was identified on the contig and subsequently the sequence eIF4G was extracted from the genome assembly. The results are summarized in table 6, below.

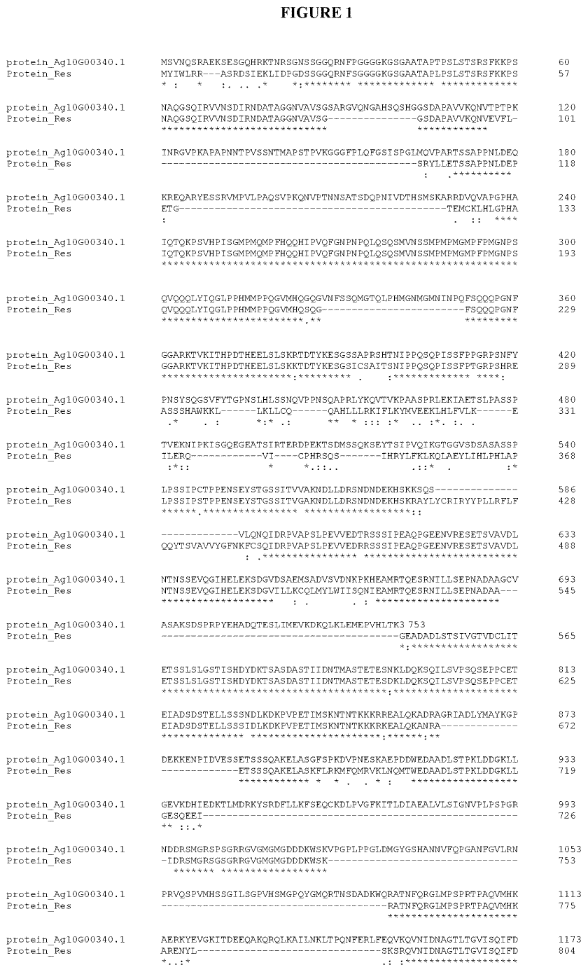

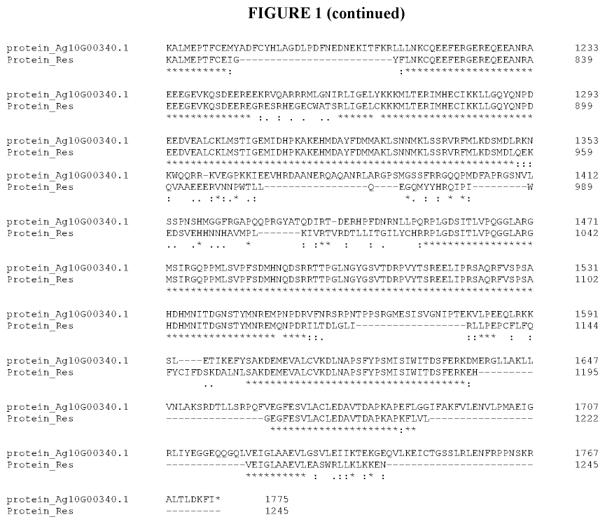

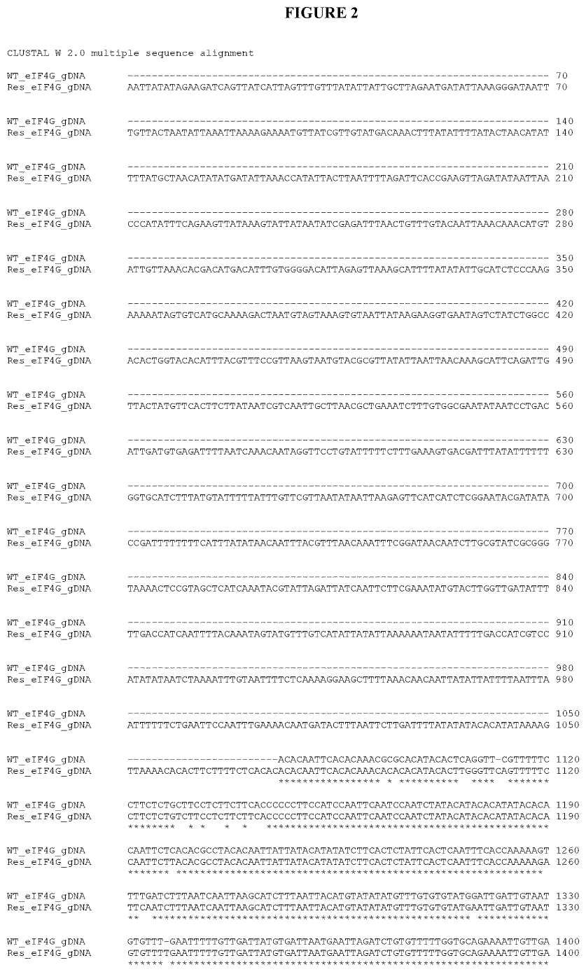

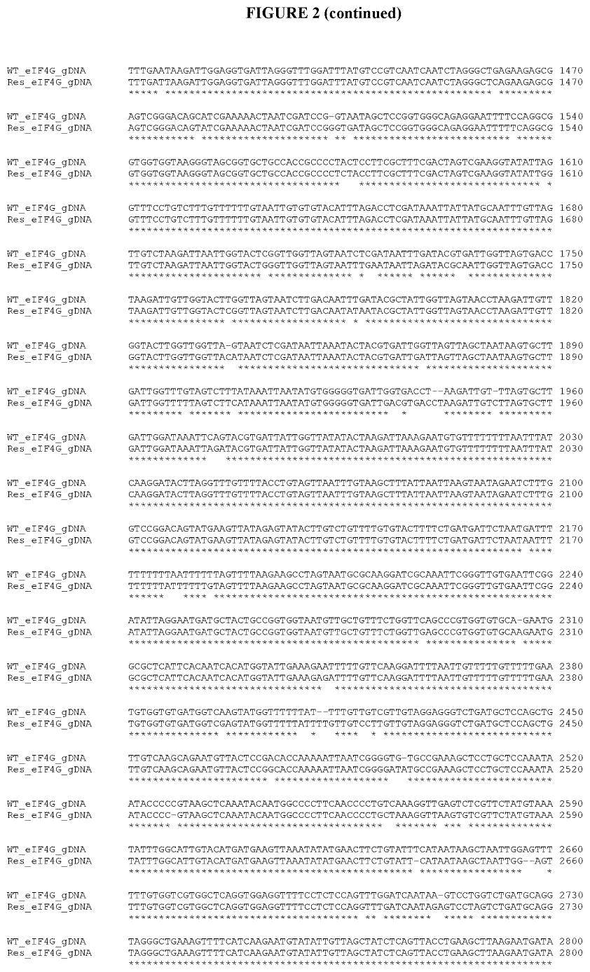

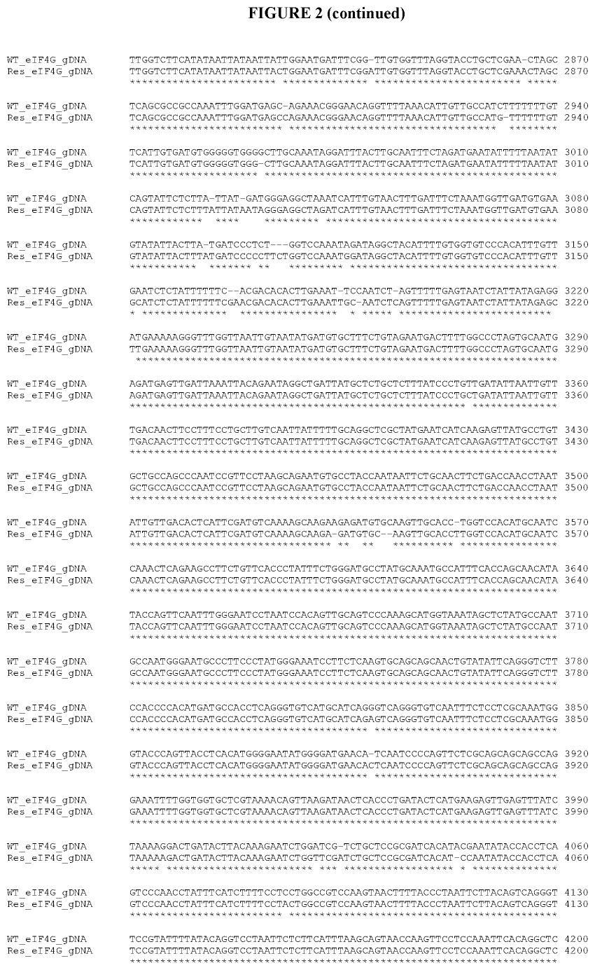

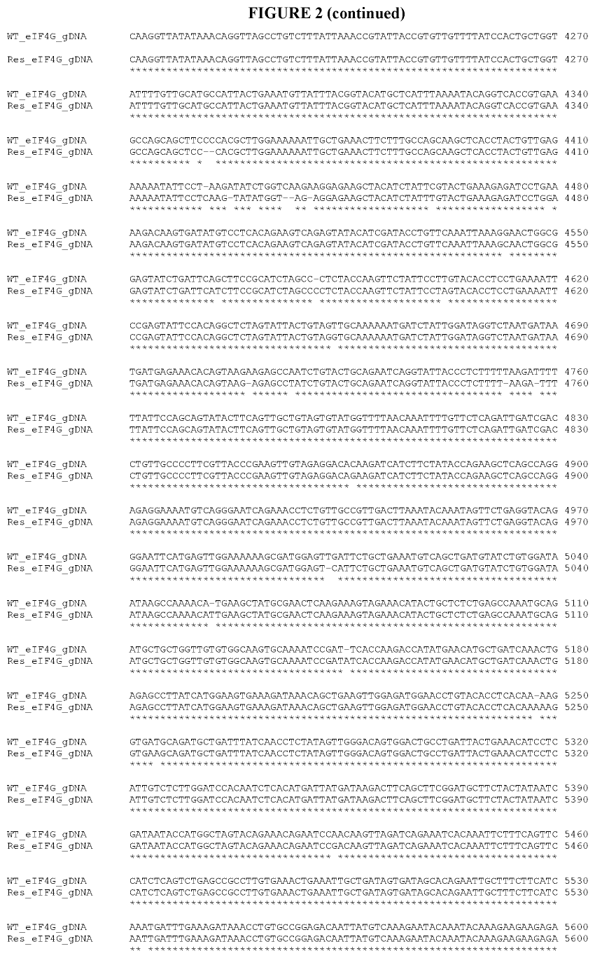

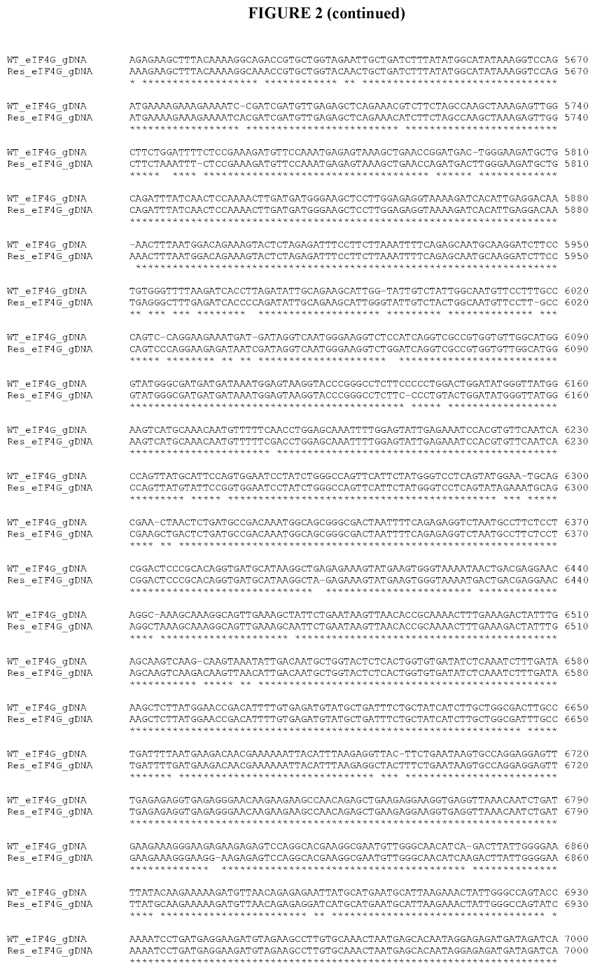

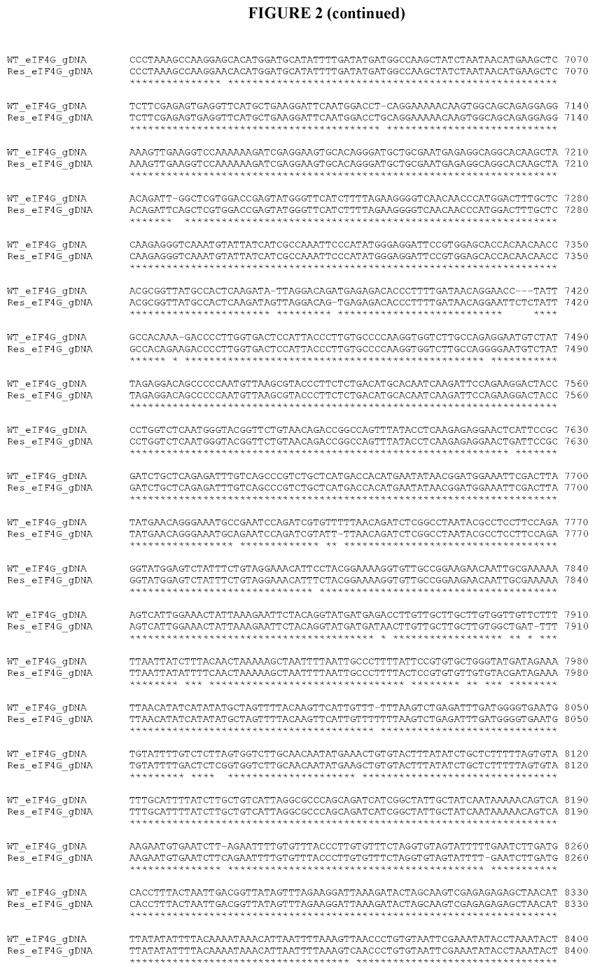

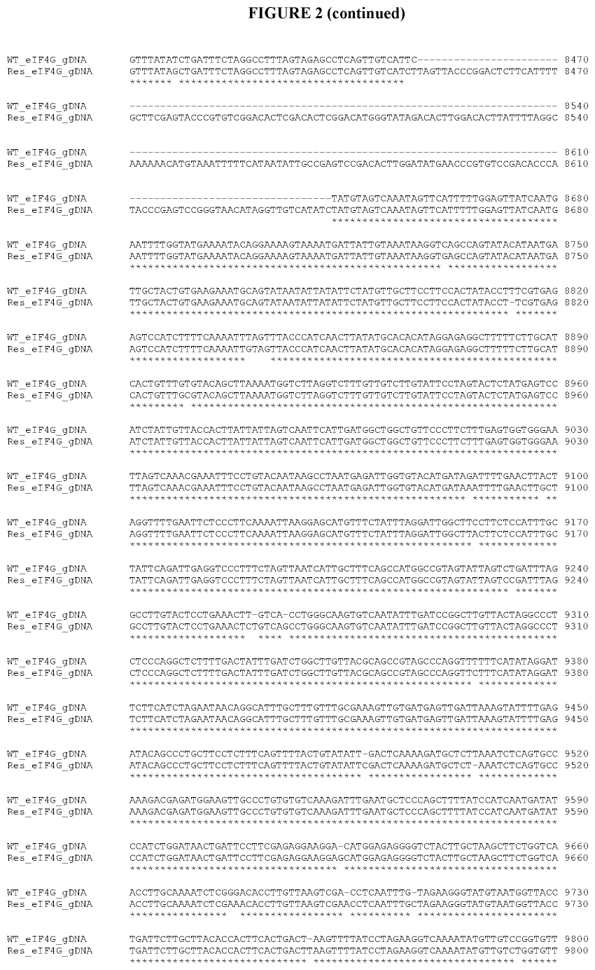

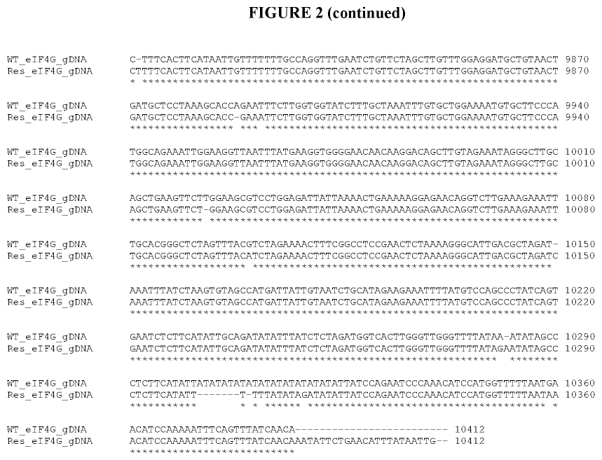

The susceptible and the resistant protein were aligned using clustalO. The alignment reveals many differences between the functional protein originating from the susceptible plants and the protein, originating from the resistant plants—see FIG. 1. The protein originating from the resistant plants is shorter (1245 amino acids) than the functional protein, originating from the susceptible plants, that has 1775 amino acids. The protein originating from the resistant plants has numerous deletions as compared to the functional protein. Examples of these deletions are —position given with reference to the sequence of the functional protein from the susceptible plants (Seq ID No 9) at amino-acids: 88-103, 120-163, 183-228, 327-350, 485-497, 691-733, 860-886, 940-994, 1022-1092, 1119-1150, 1185-1210, 1562-1580, 1690-1722 and from amino-acid 1754 forward. Additionally at least two insertions are present in the protein originating from the resistant plants, —position given with reference to the sequence of the functional protein from the susceptible plants (Seq ID No 9): at position 586 and 1593.

TABLE 5
Sequences of the susceptible plants.

| Seq | | |
|---|---|---|
| ID No | Type | Sequence |
| 7 | genomic | >eIF4G_gDNA |
| | DNA | ACACAATTCACACAAACGCGCACATACACTCAGGT |
| | susceptible | TCGTTTTTCCTTCTCTGCTTCCTCTTCTTCACCCCCC |
| | | TTCCATCCAATTCAATCCAATCTATACATACACAT |
| | | ATACACACAATTCTCACACGCCTACACAATTATTA |
| | | TACATATATCTTCACTCTATTCACTCAATTTCACCA |
| | | AAAAGTTTTGATCTTTAATCAATTAAGCATCTTTAA |
| | | TTACATGTATATATGTTTGTGTGTATGGATTGATTG |
| | | TAATGTGTTTGAATTTTTGTTGATTATGTGATTAAT |
| | | GAATTAGATCTGTGTTTTTGGTGCAGAAAATTGTT |
| | | GATTTGAATAAGATTGGAGGTGATTAGGGTTTGGA |
| | | TTTATGTCCGTCAATCAATCTAGGGCTGAGAAGAG |
| | | CGAGTCGGGACAGCATCGAAAAACTAATCGATCC |
| | | GGTAATAGCTCCGGTGGGCAGAGGAATTTTCCAGG |
| | | CGGTGGTGGTAAGGGTAGCGGTGCTGCCACCGCCC |
| | | CTACTCCTTCGCTTTCGACTAGTCGAAGGTATATTA |
| | | GGTTTCCTGTCTTTGTTTTTTGTAATTGTGTGTACA |
| | | TTTAGACCTCGATAAATTATTATGCAATTTGTTAGT |
| | | TGTCTAAGATTAATTGGTACTCGGTTGGTTAGTAA |
| | | TCTCGATAATTTGATACGTGATTGGTTAGTGACCT |
| | | AAGATTGTTGGTACTTGGTTAGTAATCTTGACAAT |
| | | TTGATACGCTATTGGTTAGTAACCTAAGATTGTTG |
| | | GTACTTGGTTGGTTAGTAATCTCGATAATTAAATA |
| | | CTACGTGATTGGTTAGTTAGCTAATAAGTGCTTGA |
| | | TTGGTTTGTAGTCTTTATAAATTAATATGTGGGGGT |
| | | GATTGGTGACCTAAGATTGTTTAGTGCTTGATTGG |
| | | ATAAATTCAGTACGTGATTATTGGTTATATACTAA |
| | | GATTAAAGAATGTGTTTTTTTTAATTTATCAAGGAT |
| | | ACTTAGGTTTGTTTTACCTGTAGTTAATTTGTAAGC |
| | | TTTATTAATTAAGTAATAGAATCTTTGGTCCGGAC |
| | | AGTATGAAGTTATAGAGTATACTTGTCTGTTTTGTG |
| | | TACTTTTCTGATGATTCTAATGATTTTTTTTTTAATT |
| | | TTTTAGTTTTAAGAAGCCTAGTAATGCGCAAGGAT |
| | | CGCAAATTCGGGTTGTGAATTCGGATATTAGGAAT |
| | | GATGCTACTGCCGGTGGTAATGTTGCTGTTTCTGGT |
| | | TCAGCCCGTGGTGTGCAGAATGGCGCTCATTCACA |
| | | ATCACATGGTATTGAAAGAATTTTTGTTCAAGGAT |
| | | TTTAATTGTTTTTGTTTTTGAATGTGGTGTGATGGT |
| | | CAAGTATGGTTTTTTATTTTGTTGTCGTTGTAGGAG |
| | | GGTCTGATGCTCCAGCTGTTGTCAAGCAGAATGTT |
| | | ACTCCGACACCAAAAATTAATCGGGGTGTGCCGAA |
| | | AGCTCCTGCTCCAAATAATACCCCCGTAAGCTCAA |
| | | ATACAATGGCCCCTTCAACCCCTGTCAAAGGTTGA |
| | | GTCTCGTTCTATGTAAATATTTGGCATTGTACATGA |
| | | TGAAGTTAAATATATGAACTTCTGTATTTCATAAT |
| | | AAGCTAATTGGAGTTTTTTGTGGTCGTGGCTCAGG |
| | | TGGAGGTTTTCCTCTCCAGTTTGGATCAATAAGTCC |
| | | TGGTCTGATGCAGGTAGGGCTGAAAGTTTTCATCA |
| | | AGAATGTATATTGTTAGCTATCTCAGTTACCTGAA |
| | | GCTTAAGAATGATATTGGTCTTCATATAATTATAA |
| | | TTATTGGAATGATTTCGGTTGTGGTTTAGGTACCTG |
| | | CTCGAACTAGCTCAGCGCCGCCAAATTTGGATGAG |
| | | CAGAAACGGGAACAGGTTTTAAACATTGTTGCCAT |
| | | CTTTTTTTGTTCATTGTGATGTGGGGGTGGGGCTTG |
| | | CAAATAGGATTTACTTGCAATTTCTAGATGAATAT |
| | | TTTTAATATCAGTATTCTCTTATTATGATGGGAGGC |
| | | TAAATCATTTGTAACTTTGATTTCTAAATGGTTGAT |
| | | GTGAAGTATATTACTTATGATCCCTCTGGTCCAAA |
| | | TAGATAGGCTACATTTTGTGGTGTCCCACATTTGTT |
| | | GAATCTCTATTTTTTCACGACACACTTGAAATTCCA |
| | | ATCTAGTTTTTGAGTAATCTATTATAGAGGATGAA |
| | | AAAGGGTTTGGTTAATTGTAATATGATGTGCTTTCT |
| | | GTAGAATGACTTTTGGCCCTAGTGCAATGAGATGA |
| | | GTTGATTAAATTACAGAATAGGCTGATTATGCTCT |
| | | GCTCTTTATCCCTGTTGATATTAATTGTTTGACAAC |
| | | TTCCTTTCCTGCTTGTCAATTATTTTTGCAGGCTCG |
| | | CTATGAATCATCAAGAGTTATGCCTGTGCTGCCAG |
| | | CCCAATCCGTTCCTAAGCAGAATGTGCCTACCAAT |
| | | AATTCTGCAACTTCTGACCAACCTAATATTGTTGA |
| | | CACTCATTCGATGTCAAAAGCAAGAAGAGATGTGC |
| | | AAGTTGCACCTGGTCCACATGCAATCCAAACTCAG |
| | | AAGCCTTCTGTTCACCCTATTTCTGGGATGCCTATG |
| | | CAAATGCCATTTCACCAGCAACATATACCAGTTCA |
| | | ATTTGGGAATCCTAATCCACAGTTGCAGTCCCAAA |
| | | GCATGGTAAATAGCTCTATGCCAATGCCAATGGGA |
| | | ATGCCCTTCCCTATGGGAAATCCTTCTCAAGTGCA |
| | | GCAGCAACTGTATATTCAGGGTCTTCCACCCCACA |
| | | TGATGCCACCTCAGGGTGTCATGCATCAGGGTCAG |
| | | GGTGTCAATTTCTCCTCGCAAATGGGTACCCAGTT |
| | | ACCTCACATGGGGAATATGGGGATGAACATCAATC |
| | | CCCAGTTCTCGCAGCAGCAGCCAGGAAATTTTGGT |
| | | GGTGCTCGTAAAACAGTTAAGATAACTCACCCTGA |
| | | TACTCATGAAGAGTTGAGTTTATCTAAAAGGACTG |
| | | ATACTTACAAAGAATCTGGATCGTCTGCTCCGCGA |
| | | TCACATACGAATATACCACCTCAGTCCCAACCTAT |
| | | TTCATCTTTTCCTCCTGGCCGTCCAAGTAACTTTTA |
| | | CCCTAATTCTTACAGTCAGGGTTCCGTATTTTATAC |
| | | AGGTCCTAATTCTCTTCATTTAAGCAGTAACCAAG |
| | | TTCCTCCAAATTCACAGGCTCCAAGGTTATATAAA |
| | | CAGGTTAGCCTGTCTTTATTAAACCGTATTACCGTG |
| | | TTGTTTTATCCACTGCTGGTATTTTGTTGCATGCCA |
| | | TTACTGAAATGTTATTTACGGTACATGCTCATTTAA |
| | | AATACAGGTCACCGTGAAGCCAGCAGCTTCCCCAC |
| | | GCTTGGAAAAAATTGCTGAAACTTCTTTGCCAGCA |
| | | AGCTCACCTACTGTTGAGAAAAATATTCCTAAGAT |
| | | ATCTGGTCAAGAAGGAGAAGCTACATCTATTCGTA |
| | | CTGAAAGAGATCCTGAAAAGACAAGTGATATGTC |
| | | CTCACAGAAGTCAGAGTATACATCGATACCTGTTC |
| | | AAATTAAAGGAACTGGCGGAGTATCTGATTCAGCT |
| | | TCCGCATCTAGCCCTCTACCAAGTTCTATTCCTTGT |
| | | ACACCTCCTGAAAATTCCGAGTATTCCACAGGCTC |
| | | TAGTATTACTGTAGTTGCAAAAAATGATCTATTGG |
| | | ATAGGTCTAATGATAATGATGAGAAACACAGTAA |
| | | GAAGAGCCAATCTGTACTGCAGAATCAGGTATTAC |
| | | CCTCTTTTTAAGATTTTTTATTCCAGCAGTATACTT |
| | | CAGTTGCTGTAGTGTATGGTTTTAACAAATTTTGTT |
| | | CTCAGATTGATCGACCTGTTGCCCCTTCGTTACCCG |
| | | AAGTTGTAGAGGACACAAGATCATCTTCTATACCA |
| | | GAAGCTCAGCCAGGAGAGGAAAATGTCAGGGAAT |
| | | CAGAAACCTCTGTTGCCGTTGACTTAAATACAAAT |
| | | AGTTCTGAGGTACAGGGAATTCATGAGTTGGAAAA |
| | | AAGCGATGGAGTTGATTCTGCTGAAATGTCAGCTG |
| | | ATGTATCTGTGGATAATAAGCCAaaacatgaagct |
| | | atgcgaactcaagaaagtagaaacatactgctctc |
| | | tgagccaaatgcagatgctgctggttgtgtggcaa |
| | | gtgcaaaatccgattcACCAAGACCATATGAACATG |
| | | CTGATCAAACTGAGAGCCTTATCATGGAAGTGAAAG |
| | | ATAAACAGCTGAAGTTGGAGATGGAACCTGTACA |
| | | CCTCACAAAAGGTGATGCAGATGCTGATTTATCAA |
| | | CCTCTATAGTTGGGACAGTGGACTGCCTGATTACT |
| | | GAAACATCCTCATTGTCTCTTGGATCCACAATCTC |
| | | ACATGATTATGATAAGACTTCAGCTTCGGATGCTT |
| | | CTACTATAATCGATAATACCATGGCTAGTACAGAA |
| | | ACAGAATCCAACAAGTTAGATCAGAAATCACAAA |
| | | TTCTTTCAGTTCCATCTCAGTCTGAGCCGCCTTGTG |
| | | AAACTGAAATTGCTGATAGTGATAGCACAGAATTG |
| | | CTTTCTTCATCAAATGATTTGAAAGATAAACCTGT |
| | | GCCGGAGACAATTATGTCAAAGAATACAAATACA |
| | | AAGAAGAAGAGAAGAGAAGCTTTACAAAAGGCAG |
| | | ACCGTGCTGGTAGAATTGCTGATCTTTATATGGCA |
| | | TATAAAGGTCCAGATGAAAAGAAAGAAAATCCGA |
| | | TCGATGTTGAGAGCTCAGAAACGTCTTCTAGCCAA |
| | | GCTAAAGAGTTGGCTTCTGGATTTTCTCCGAAAGA |
| | | TGTTCCAAATGAGAGTAAAGCTGAACCGGATGACT |
| | | GGGAAGATGCTGCAGATTTATCAACTCCAAAACTT |
| | | GATGATGGGAAGCTCCTTGGAGAGGTAAAAGATC |
| | | ACATTGAGGACAAAACTTTAATGGACAGAAAGTA |
| | | CTCTAGAGATTTCCTTCTTAAATTTTCAGAGCAATG |
| | | CAAGGATCTTCCTGTGGGTTTTAAGATCACCTTAG |
| | | ATATTGCAGAAGCATTGGTATTGTCTATTGGCAAT |
| | | GTTCCTTTGCCCAGTCCAGGAAGAAATGATGATAG |
| | | GTCAATGGGAAGGTCTCCATCAGGTCGCCGTGGTG |
| | | TTGGCATGGGTATGGGCGATGATGATAAATGGAGT |
| | | AAGGTACCCGGGCCTCTTCCCCCTGGACTGGATAT |
| | | GGGTTATGGAAGTCATGCAAACAATGTTTTTCAAC |
| | | CTGGAGCAAATTTTGGAGTATTGAGAAATCCACGT |
| | | GTTCAATCACCAGTTATGCATTCCAGTGGAATCCT |
| | | ATCTGGGCCAGTTCATTCTATGGGTCCTCAGTATG |
| | | GAATGCAGCGAACTAACTCTGATGCCGACAAATG |
| | | GCAGCGGGCGACTAATTTTCAGAGAGGTCTAATGC |
| | | CTTCTCCTCGGACTCCCGCACAGGTGATGCATAAG |
| | | GCTGAGAGAAAGTATGAAGTGGGTAAAATAACTG |
| | | ACGAGGAACAGGCAAAGCAAAGGCAGTTGAAAGC |
| | | TATTCTGAATAAGTTAACACCGCAAAACTTTGAAA |
| | | GACTATTTGAGCAAGTCAAGCAAGTAAATATTGAC |
| | | AATGCTGGTACTCTCACTGGTGTGATATCTCAAAT |
| | | CTTTGATAAAGCTCTTATGGAACCGACATTTTGTG |
| | | AGATGTATGCTGATTTCTGCTATCATCTTGCTGGCG |
| | | ACTTGCCTGATTTTAATGAAGACAACGAAAAAATT |
| | | ACATTTAAGAGGTTACTTCTGAATAAGTGCCAGGA |
| | | GGAGTTTGAGAGAGGTGAGAGGGAACAAGAAGAA |
| | | GCCAACAGAGCTGAAGAGGAAGGTGAGGTTAAAC |
| | | AATCTGATGAAGAAAGGGAAGAGAAGAGAGTCCA |
| | | GGCACGAAGGCGAATGTTGGGCAACATCAGACTT |
| | | ATTGGGGAATTATACAAGAAAAAGATGTTAACAG |
| | | AGAGAATTATGCATGAATGCATTAAGAAACTATTG |
| | | GGCCAGTACCAAAATCCTGATGAGGAAGATGTAG |
| | | AAGCCTTGTGCAAACTAATGAGCACAATAGGAGA |
| | | GATGATAGATCACCCTAAAGCCAAGGAGCACATG |
| | | GATGCATATTTTGATATGATGGCCAAGCTATCTAA |
| | | TAACATGAAGCTCTCTTCGAGAGTGAGGTTCATGC |
| | | TGAAGGATTCAATGGACCTCAGGAAAAACAAGTG |
| | | GCAGCAGAGGAGGAAAGTTGAAGGTCCAAAAAAG |
| | | ATCGAGGAAGTGCACAGGGATGCTGCGAATGAGA |
| | | GGCAGGCACAAGCTAACAGATTGGCTCGTGGACC |
| | | GAGTATGGGTTCATCTTTTAGAAGGGGTCAACAAC |
| | | CCATGGACTTTGCTCCAAGAGGGTCAAATGTATTA |
| | | TCATCGCCAAATTCCCATATGGGAGGATTCCGTGG |
| | | AGCACCACAACAACCACGCGGTTATGCCACTCAAG |
| | | ATATTAGGACAGATGAGAGACACCCTTTTGATAAC |
| | | AGGAACCTATTGCCACAAAGACCCCTTGGTGACTC |
| | | CATTACCCTTGTGCCCCAAGGTGGTCTTGCCAGAG |
| | | GAATGTCTATTAGAGGACAGCCCCCAATGTTAAGC |
| | | GTACCCTTCTCTGACATGCACAATCAAGATTCCAG |
| | | AAGGACTACCCCTGGTCTCAATGGGTACGGTTCTG |
| | | TAACAGACCGGCCAGTTTATACCTCAAGAGAGGA |
| | | ACTCATTCCGCGATCTGCTCAGAGATTTGTCAGCC |
| | | CGTCTGCTCATGACCACATGAATATAACGGATGGA |
| | | AATTCGACTTATATGAACAGGGAAATGCCGAATCC |
| | | AGATCGTGTTTTTAACAGATCTCGGCCTAATACGC |
| | | CTCCTTCCAGAGGTATGGAGTCTATTTCTGTAGGA |
| | | AACATTCCTACGGAAAAGGTGTTGCCGGAAGAAC |
| | | AATTGCGAAAAAAGTCATTGGAAACTATTAAAGA |
| | | ATTCTACAGGTATGATGAGACCTTGTTGCTTGCTTG |
| | | TGGTTGTTCTTTTTAATTATCTTTACAACTAAAAAG |
| | | CTAATTTTAATTGCCCTTTTATTCCGTGTGCTGGGT |
| | | ATGATAGAAATTAACATATCATATATGCTAGTTTT |
| | | ACAAGTTCATTGTTTTTTAAGTCTGAGATTTGATGG |
| | | GGTGAATGTGTATTTTGTCTCTTAGTGGTCTTGCAA |
| | | CAATATGAAACTGTGTACTTTATATCTGCTCTTTTT |
| | | AGTGTATTTGCATTTTATCTTGCTGTCATTAGGCGC |
| | | CCAGCAGATCATCGGCTATTGCTATCAATAAAAAC |
| | | AGTCAAAGAATGTGAATCTTAGAATTTTGTGTTTA |
| | | CCCTTGTGTTTCTAGGTGTAGTATTTTTGAATCTTG |
| | | ATGCACCTTTACTAATTGACGGTTATAGTTTAGAA |
| | | GGATTAAAGATACTAGCAAGTCGAGAGAGAGCTA |
| | | ACATTTATATATTTTACAAAATAAACATTAATTTTA |
| | | AAGTTAACCCTGTGTAATTCGAAATATACCTAAAT |
| | | ACTGTTTATATCTGATTTCTAGGCCTTTAGTAGAGC |
| | | CTCAGTTGTCATTCTATGTAGTCAAATAGTTCATTT |
| | | TTGGAGTTATCAATGAATTTTGGTATGAAAATACA |
| | | GGAAAAGTAAAATGATTATTGTAAATAAGGTCAG |
| | | CCAGTATACATAATGATTGCTACTGTGAAGAAATG |
| | | CAGTATAATATTATATTCTATGTTGCTTCCTTCCAC |
| | | TATACCTTTCGTGAGAGTCCATCTTTTCAAAATTTA |
| | | GTTTACCCATCAACTTATATGCACACATAGGAGAG |
| | | GCTTTTTCTTGCATCACTGTTTGTGTACAGCTTAAA |
| | | ATGGTCTTAGGTCTTTGTTGTCTTGTATTCCTAGTA |
| | | CTCTATGAGTCCATCTATTGTTACCACTTATTATTA |
| | | GTCAATTCATTGATGGCTGGCTGTTCCCTTCTTTGA |
| | | GTGGTGGGAATTAGTCAAACGAAATTTCCTGTACA |
| | | ATAAGCCTAATGAGATTGGTGTACATGATAGATTT |
| | | TGAACTTACTAGGTTTTGAATTCTCCCTTCAAAATT |
| | | AAGGAGCATGTTTCTATTTAGGATTGGCTTCCTTCT |
| | | CCATTTGCTATTCAGATTGAGGTCCCTTTCTAGTTA |
| | | ATCATTGCTTTCAGCCATGGCCGTAGTATTAGTCTG |
| | | ATTTAGGCCTTGTACTCCTGAAACTTGTCACCTGG |
| | | GCAAGTGTCAATATTTGATCCGGCTTGTTACTAGG |
| | | CCCTCTCCCAGGCTCTTTTGACTATTTGATCTGGCT |
| | | TGTTACGCAGCCGTAGCCCAGGTTTTTTCATATAG |
| | | GATTCTTCATCTAGAATAACAGGCATTTGCTTTGTT |
| | | TGCGAAAGTTGTGATGAGTTGATTAAAGTATTTTG |
| | | AGATACAGCCCTGCTTCCTCTTTCAGTTTTACTGTA |
| | | TATTGACTCAAAAGATGCTCTTAAATCTCAGTGCC |
| | | AAAGACGAGATGGAAGTTGCCCTGTGTGTCAAAG |
| | | ATTTGAATGCTCCCAGCTTTTATCCATCAATGATAT |
| | | CCATCTGGATAACTGATTCCTTCGAGAGGAAGGAC |
| | | ATGGAGAGGGGTCTACTTGCTAAGCTTCTGGTCAA |
| | | CCTTGCAAAATCTCGGGACACCTTGTTAAGTCGAC |
| | | CTCAATTTGTAGAAGGGTATGTAATGGTTACCTGA |
| | | TTCTTGCTTACACCACTTCACTGACTAAGTTTTATC |
| | | CTAGAAGGTCAAAATATGTTGTCCGGTGTTCTTTC |
| | | ACTTCATAATTGTTTTTTTGCCAGGTTTGAATCTGT |
| | | TCTAGCTTGTTTGGAGGATGCTGTAACTGATGCTC |
| | | CTAAAGCACCAGAATTTCTTGGTGGTATCTTTGCT |
| | | AAATTTGTGCTGGAAAATGTGCTTCCCATGGCAGA |
| | | AATTGGAAGGTTAATTTATGAAGGTGGGGAACAA |
| | | CAAGGACAGCTTGTAGAAATAGGGCTTGCAGCTG |
| | | AAGTTCTTGGAAGCGTCCTGGAGATTATTAAAACT |
| | | GAAAAAGGAGAACAGGTCTTGAAAGAAATTTGCA |
| | | CGGGCTCTAGTTTACGTCTAGAAAACTTTCGGCCT |
| | | CCGAACTCTAAAAGGGCATTGACGCTAGATAAATT |
| | | TATCTAAGTGTAGCCATGATTATTGTAATCTGCAT |
| | | AGAAGAAATTTTATGTCCAGCCCTATCAGTGAATC |
| | | TCTTCATATTGcagatatatttatctctagatggt |
| | | cacttgggttgggttttataaatatagccctcttc |
| | | atattatatatatatatatatatatatattatcca |
| | | gaatcccaaacatccatggtttttaatgaaCATCC |
| | | AAAAATTTCAGTTTATCAACA |
| 8 | cDNA | >eIF4G_cDNA |
| | susceptible | ATGTCCGTCAATCAATCTAGGGCTGAGAAGAGCGA |
| | | GTCGGGACAGCATCGAAAAACTAATCGATCCGGT |
| | | AATAGCTCCGGTGGGCAGAGGAATTTTCCAGGCGG |
| | | TGGTGGTAAGGGTAGCGGTGCTGCCACCGCCCCTA |
| | | CTCCTTCGCTTTCGACTAGTCGAAGTTTTAAGAAG |
| | | CCTAGTAATGCGCAAGGATCGCAAATTCGGGTTGT |
| | | GAATTCGGATATTAGGAATGATGCTACTGCCGGTG |
| | | GTAATGTTGCTGTTTCTGGTTCAGCCCGTGGTGTGC |
| | | AGAATGGCGCTCATTCACAATCACATGGAGGGTCT |
| | | GATGCTCCAGCTGTTGTCAAGCAGAATGTTACTCC |
| | | GACACCAAAAATTAATCGGGGTGTGCCGAAAGCT |
| | | CCTGCTCCAAATAATACCCCCGTAAGCTCAAATAC |
| | | AATGGCCCCTTCAACCCCTGTCAAAGGTGGAGGTT |
| | | TTCCTCTCCAGTTTGGATCAATAAGTCCTGGTCTGA |
| | | TGCAGGTACCTGCTCGAACTAGCTCAGCGCCGCCA |
| | | AATTTGGATGAGCAGAAACGGGAACAGGCTCGCT |
| | | ATGAATCATCAAGAGTTATGCCTGTGCTGCCAGCC |
| | | CAATCCGTTCCTAAGCAGAATGTGCCTACCAATAA |
| | | TTCTGCAACTTCTGACCAACCTAATATTGTTGACAC |
| | | TCATTCGATGTCAAAAGCAAGAAGAGATGTGCAA |
| | | GTTGCACCTGGTCCACATGCAATCCAAACTCAGAA |
| | | GCCTTCTGTTCACCCTATTTCTGGGATGCCTATGCA |
| | | AATGCCATTTCACCAGCAACATATACCAGTTCAAT |
| | | TTGGGAATCCTAATCCACAGTTGCAGTCCCAAAGC |
| | | ATGGTAAATAGCTCTATGCCAATGCCAATGGGAAT |
| | | GCCCTTCCCTATGGGAAATCCTTCTCAAGTGCAGC |
| | | AGCAACTGTATATTCAGGGTCTTCCACCCCACATG |
| | | ATGCCACCTCAGGGTGTCATGCATCAGGGTCAGGG |
| | | TGTCAATTTCTCCTCGCAAATGGGTACCCAGTTAC |
| | | CTCACATGGGGAATATGGGGATGAACATCAATCCC |
| | | CAGTTCTCGCAGCAGCAGCCAGGAAATTTTGGTGG |
| | | TGCTCGTAAAACAGTTAAGATAACTCACCCTGATA |
| | | CTCATGAAGAGTTGAGTTTATCTAAAAGGACTGAT |
| | | ACTTACAAAGAATCTGGATCGTCTGCTCCGCGATC |
| | | ACATACGAATATACCACCTCAGTCCCAACCTATTT |
| | | CATCTTTTCCTCCTGGCCGTCCAAGTAACTTTTACC |
| | | CTAATTCTTACAGTCAGGGTTCCGTATTTTATACAG |
| | | GTCCTAATTCTCTTCATTTAAGCAGTAACCAAGTTC |
| | | CTCCAAATTCACAGGCTCCAAGGTTATATAAACAG |
| | | GTCACCGTGAAGCCAGCAGCTTCCCCACGCTTGGA |
| | | AAAAATTGCTGAAACTTCTTTGCCAGCAAGCTCAC |
| | | CTACTGTTGAGAAAAATATTCCTAAGATATCTGGT |
| | | CAAGAAGGAGAAGCTACATCTATTCGTACTGAAA |
| | | GAGATCCTGAAAAGACAAGTGATATGTCCTCACAG |
| | | AAGTCAGAGTATACATCGATACCTGTTCAAATTAA |
| | | AGGAACTGGCGGAGTATCTGATTCAGCTTCCGCAT |
| | | CTAGCCCTCTACCAAGTTCTATTCCTTGTACACCTC |
| | | CTGAAAATTCCGAGTATTCCACAGGCTCTAGTATT |
| | | ACTGTAGTTGCAAAAAATGATCTATTGGATAGGTC |
| | | TAATGATAATGATGAGAAACACAGTAAGAAGAGC |
| | | CAATCTGTACTGCAGAATCAGATTGATCGACCTGT |
| | | TGCCCCTTCGTTACCCGAAGTTGTAGAGGACACAA |
| | | GATCATCTTCTATACCAGAAGCTCAGCCAGGAGAG |
| | | GAAAATGTCAGGGAATCAGAAACCTCTGTTGCCGT |
| | | TGACTTAAATACAAATAGTTCTGAGGTACAGGGAA |
| | | TTCATGAGTTGGAAAAAAGCGATGGAGTTGATTCT |
| | | GCTGAAATGTCAGCTGATGTATCTGTGGATAATAA |
| | | GCCAAAACATGAAGCTATGCGAACTCAAGAAAGT |
| | | AGAAACATACTGCTCTCTGAGCCAAATGCAGATGC |
| | | TGCTGGTTGTGTGGCAAGTGCAAAATCCGATTCAC |
| | | CAAGACCATATGAACATGCTGATCAAACTGAGAG |
| | | CCTTATCATGGAAGTGAAAGATAAACAGCTGAAGT |
| | | TGGAGATGGAACCTGTACACCTCACAAAAGGTGAT |
| | | GCAGATGCTGATTTATCAACCTCTATAGTTGGGAC |
| | | AGTGGACTGCCTGATTACTGAAACATCCTCATTGT |
| | | CTCTTGGATCCACAATCTCACATGATTATGATAAG |
| | | ACTTCAGCTTCGGATGCTTCTACTATAATCGATAAT |
| | | ACCATGGCTAGTACAGAAACAGAATCCAACAAGT |
| | | TAGATCAGAAATCACAAATTCTTTCAGTTCCATCT |
| | | CAGTCTGAGCCGCCTTGTGAAACTGAAATTGCTGA |
| | | TAGTGATAGCACAGAATTGCTTTCTTCATCAAATG |
| | | ATTTGAAAGATAAACCTGTGCCGGAGACAATTATG |
| | | TCAAAGAATACAAATACAAAGAAGAAGAGAAGAG |
| | | AAGCTTTACAAAAGGCAGACCGTGCTGGTAGAATT |
| | | GCTGATCTTTATATGGCATATAAAGGTCCAGATGA |
| | | AAAGAAAGAAAATCCGATCGATGTTGAGAGCTCA |
| | | GAAACGTCTTCTAGCCAAGCTAAAGAGTTGGCTTC |
| | | TGGATTTTCTCCGAAAGATGTTCCAAATGAGAGTA |
| | | AAGCTGAACCGGATGACTGGGAAGATGCTGCAGA |
| | | TTTATCAACTCCAAAACTTGATGATGGGAAGCTCC |
| | | TTGGAGAGGTAAAAGATCACATTGAGGACAAAAC |
| | | TTTAATGGACAGAAAGTACTCTAGAGATTTCCTTC |
| | | TTAAATTTTCAGAGCAATGCAAGGATCTTCCTGTG |
| | | GGTTTTAAGATCACCTTAGATATTGCAGAAGCATT |
| | | GGTATTGTCTATTGGCAATGTTCCTTTGCCCAGTCC |
| | | AGGAAGAAATGATGATAGGTCAATGGGAAGGTCT |
| | | CCATCAGGTCGCCGTGGTGTTGGCATGGGTATGGG |
| | | CGATGATGATAAATGGAGTAAGGTACCCGGGCCTC |
| | | TTCCCCCTGGACTGGATATGGGTTATGGAAGTCAT |
| | | GCAAACAATGTTTTTCAACCTGGAGCAAATTTTGG |
| | | AGTATTGAGAAATCCACGTGTTCAATCACCAGTTA |
| | | TGCATTCCAGTGGAATCCTATCTGGGCCAGTTCAT |
| | | TCTATGGGTCCTCAGTATGGAATGCAGCGAACTAA |
| | | CTCTGATGCCGACAAATGGCAGCGGGCGACTAATT |
| | | TTCAGAGAGGTCTAATGCCTTCTCCTCGGACTCCC |
| | | GCACAGGTGATGCATAAGGCTGAGAGAAAGTATG |
| | | AAGTGGGTAAAATAACTGACGAGGAACAGGCAAA |
| | | GCAAAGGCAGTTGAAAGCTATTCTGAATAAGTTAA |
| | | CACCGCAAAACTTTGAAAGACTATTTGAGCAAGTC |
| | | AAGCAAGTAAATATTGACAATGCTGGTACTCTCAC |
| | | TGGTGTGATATCTCAAATCTTTGATAAAGCTCTTAT |
| | | GGAACCGACATTTTGTGAGATGTATGCTGATTTCT |
| | | GCTATCATCTTGCTGGCGACTTGCCTGATTTTAATG |
| | | AAGACAACGAAAAAATTACATTTAAGAGGTTACTT |
| | | CTGAATAAGTGCCAGGAGGAGTTTGAGAGAGGTG |
| | | AGAGGGAACAAGAAGAAGCCAACAGAGCTGAAG |
| | | AGGAAGGTGAGGTTAAACAATCTGATGAAGAAAG |
| | | GGAAGAGAAGAGAGTCCAGGCACGAAGGCGAATG |
| | | TTGGGCAACATCAGACTTATTGGGGAATTATACAA |
| | | GAAAAAGATGTTAACAGAGAGAATTATGCATGAA |
| | | TGCATTAAGAAACTATTGGGCCAGTACCAAAATCC |
| | | TGATGAGGAAGATGTAGAAGCCTTGTGCAAACTA |
| | | ATGAGCACAATAGGAGAGATGATAGATCACCCTA |
| | | AAGCCAAGGAGCACATGGATGCATATTTTGATATG |
| | | ATGGCCAAGCTATCTAATAACATGAAGCTCTCTTC |
| | | GAGAGTGAGGTTCATGCTGAAGGATTCAATGGACC |
| | | TCAGGAAAAACAAGTGGCAGCAGAGGAGGAAAGT |
| | | TGAAGGTCCAAAAAAGATCGAGGAAGTGCACAGG |
| | | GATGCTGCGAATGAGAGGCAGGCACAAGCTAACA |
| | | GATTGGCTCGTGGACCGAGTATGGGTTCATCTTTT |
| | | AGAAGGGGTCAACAACCCATGGACTTTGCTCCAAG |
| | | AGGGTCAAATGTATTATCATCGCCAAATTCCCATA |
| | | TGGGAGGATTCCGTGGAGCACCACAACAACCACG |
| | | CGGTTATGCCACTCAAGATATTAGGACAGATGAGA |
| | | GACACCCTTTTGATAACAGGAACCTATTGCCACAA |
| | | AGACCCCTTGGTGACTCCATTACCCTTGTGCCCCA |
| | | AGGTGGTCTTGCCAGAGGAATGTCTATTAGAGGAC |
| | | AGCCCCCAATGTTAAGCGTACCCTTCTCTGACATG |
| | | CACAATCAAGATTCCAGAAGGACTACCCCTGGTCT |
| | | CAATGGGTACGGTTCTGTAACAGACCGGCCAGTTT |
| | | ATACCTCAAGAGAGGAACTCATTCCGCGATCTGCT |
| | | CAGAGATTTGTCAGCCCGTCTGCTCATGACCACAT |
| | | GAATATAACGGATGGAAATTCGACTTATATGAACA |
| | | GGGAAATGCCGAATCCAGATCGTGTTTTTAACAGA |
| | | TCTCGGCCTAATACGCCTCCTTCCAGAGGTATGGA |
| | | GTCTATTTCTGTAGGAAACATTCCTACGGAAAAGG |
| | | TGTTGCCGGAAGAACAATTGCGAAAAAAGTCATTG |
| | | GAAACTATTAAAGAATTCTACAGTGCCAAAGACG |
| | | AGATGGAAGTTGCCCTGTGTGTCAAAGATTTGAAT |
| | | GCTCCCAGCTTTTATCCATCAATGATATCCATCTGG |
| | | ATAACTGATTCCTTCGAGAGGAAGGACATGGAGA |
| | | GGGGTCTACTTGCTAAGCTTCTGGTCAACCTTGCA |
| | | AAATCTCGGGACACCTTGTTAAGTCGACCTCAATT |
| | | TGTAGAAGGGTTTGAATCTGTTCTAGCTTGTTTGG |
| | | AGGATGCTGTAACTGATGCTCCTAAAGCACCAGAA |
| | | TTTCTTGGTGGTATCTTTGCTAAATTTGTGCTGGAA |
| | | AATGTGCTTCCCATGGCAGAAATTGGAAGGTTAAT |
| | | TTATGAAGGTGGGGAACAACAAGGACAGCTTGTA |
| | | GAAATAGGGCTTGCAGCTGAAGTTCTTGGAAGCGT |
| | | CCTGGAGATTATTAAAACTGAAAAAGGAGAACAG |
| | | GTCTTGAAAGAAATTTGCACGGGCTCTAGTTTACG |
| | | TCTAGAAAACTTTCGGCCTCCGAACTCTAAAAGGG |
| | | CATTGACGCTAGATAAATTTATCTAA |
| Seq | Protein, | >protein_Ag10G00340.1 |
| IND no | susceptible | MSVNQSRAEKSESGQHRKTNRSGNSSGGQRNFPGGGGKG |
| 9 | | SGAATAPTPSLSTSRSFKKPSNAQGSQIRVVNSDIRNDATA |
| | | GGNVAVSGSARGVQNGAHSQSHGGSDAPAVVKQNVTPTP |
| | | KINRGVPKAPAPNNTPVSSNTMAPSTPVKGGGFPLQFGSIS |
| | | PGLMQVPARTSSAPPNLDEQKREQARYESSRVMPVLPAQS |
| | | VPKQNVPTNNSATSDQPNIVDTHSMSKARRDVQVAPGPH |
| | | AIQTQKPSVHPISGMPMQMPFHQQHIPVQFGNPNPQLQSQ |
| | | SMVNSSMPMPMGMPFPMGNPSQVQQQLYIQGLPPHMMP |
| | | PQGVMHQGQGVNFSSQMGTQLPHMGNMGMNINPQFSQQ |
| | | QPGNFGGARKTVKITHPDTHEELSLSKRTDTYKESGSSAPR |
| | | SHTNIPPQSQPISSFPPGRPSNFYPNSYSQGSVFYTGPNSLHL |
| | | SSNQVPPNSQAPRLYKQVTVKPAASPRLEKIAETSLPASSP |
| | | TVEKNIPKISGQEGEATSIRTERDPEKTSDMSSQKSEYTSIP |
| | | VQIKGTGGVSDSASASSPLPSSIPCTPPENSEYSTGSSITVVA |
| | | KNDLLDRSNDNDEKHSKKSQSVLQNQIDRPVAPSLPEVVE |
| | | DTRSSSIPEAQPGEENVRESETSVAVDLNTNSSEVQGIHELE |
| | | KSDGVDSAEMSADVSVDNKPKHEAMRTQESRNILLSEPN |
| | | ADAAGCVASAKSDSPRPYEHADQTESLIMEVKDKQLKLE |
| | | MEPVHLTKGDADADLSTSIVGTVDCLITETSSLSLGSTISHD |
| | | YDKTSASDASTIIDNTMASTETESNKLDQKSQILSVPSQSEP |
| | | PCETEIADSDSTELLSSSNDLKDKPVPETIMSKNTNTKKKR |
| | | REALQKADRAGRIADLYMAYKGPDEKKENPIDVESSETSS |
| | | SQAKELASGFSPKDVPNESKAEPDDWEDAADLSTPKLDDG |
| | | KLLGEVKDHIEDKTLMDRKYSRDFLLKFSEQCKDLPVGFK |
| | | ITLDIAEALVLSIGNVPLPSPGRNDDRSMGRSPSGRRGVGM |
| | | GMGDDDKWSKVPGPLPPGLDMGYGSHANNVFQPGANFG |
| | | VLRNPRVQSPVMHSSGILSGPVHSMGPQYGMQRTNSDAD |
| | | KWQRATNFQRGLMPSPRTPAQVMHKAERKYEVGKITDEE |
| | | QAKQRQLKAILNKLTPQNFERLFEQVKQVNIDNAGTLTGV |
| | | ISQIFDKALMEPTFCEMYADFCYHLAGDLPDFNEDNEKITF |
| | | KRLLLNKCQEEFERGEREQEEANRAEEEGEVKQSDEEREE |
| | | KRVQARRRMLGNIRLIGELYKKKMLTERIMHECIKKLLGQ |
| | | YQNPDEEDVEALCKLMSTIGEMIDHPKAKEHMDAYFDM |
| | | MAKLSNNMKLSSRVRFMLKDSMDLRKNKWQQRRKVEGP |
| | | KKIEEVHRDAANERQAQANRLARGPSMGSSFRRGQQPMD |
| | | FAPRGSNVLSSPNSHMGGFRGAPQQPRGYATQDIRTDERH |
| | | PFDNRNLLPQRPLGDSITLVPQGGLARGMSIRGQPPMLSVP |
| | | FSDMHNQDSRRTTPGLNGYGSVTDRPVYTSREELIPRSAQ |
| | | RFVSPSAHDHMNITDGNSTYMNREMPNPDRVFNRSRPNTP |
| | | PSRGMESISVGNIPTEKVLPEEQLRKKSLETIKEFYSAKDEM |
| | | EVALCVKDLNAPSFYPSMISIWITDSFERKDMERGLLAKLL |
| | | VNLAKSRDTLLSRPQFVEGFESVLACLEDAVTDAPKAPEF |
| | | LGGIFAKFVLENVLPMAEIGRLIYEGGEQQGQLVEIGLAAE |
| | | VLGSVLEIIKTEKGEQVLKEICTGSSLRLENFRPPNSKRALT |
| | | LDKFI* |

TABLE 6
Sequences of the resistant plants.

| Seq | | |
|---|---|---|
| ID No | Type | Sequence |
| 10 | genomic | >Res_gDNA |
| | DNA | CAATTATAAATGTTCAGAATATTTGTTGATAAACT |
| | resistant | GAAATTTTTGGATGTTTATTAAAAACCATGGATGT |
| | | TTGGGATTCTGGATAATATATCTATATAAAAAATA |
| | | TGAAGAGGGCTATATTCTATAAAACCCAACCCAAG |
| | | TGACCATCTAGAGATAAATATATCTGCAATATGAA |
| | | GAGATTCACTGATAGGGCTGGACATAAAATTTCTT |
| | | CTATGCAGATTACAATAATCATGGCTACACTTAGA |
| | | TAAATTTGATCTAGCGTCAATGCCCTTTTAGAGTTC |
| | | GGAGGCCGAAAGTTTTCTAGATGTAAACTAGAGCC |
| | | CGTGCAAATTTCTTTCAAGACCTGTTCTCCTTTTTC |
| | | AGTTTTAATAATCTCCAGGACGCTTCCAGAACTTC |
| | | AGCTGCAAGCCCTATTTCTACAAGCTGTCCTTGTTG |
| | | TTCCCCACCTTCATAAATTAACCTTCCAATTTCTGC |
| | | CATGGGAAGCACATTTTCCAGCACAAATTTAGCAA |
| | | AGATACCACCAAGAATTTCGGTGCTTTAGGAGCAT |
| | | CAGTTACAGCATCCTCCAAACAAGCTAGAACAGAT |
| | | TCAAACCTGGCAAAAAAACAATTATGAAGTGAAA |
| | | AGAACACCAGACAACATATTTTGACCTTCTAGGAT |
| | | AAAACTTAAGTCAGTGAAGTGGTGTAAGCAAGAA |
| | | TCAGGTAACCATTACATACCCTTCTAGCAAATTGA |
| | | GGTTCGACTTAACAAGGTGTTTCGAGATTTTGCAA |
| | | GGTTGACCAGAAGCTTAGCAAGTAGACCCCTCTCC |
| | | ATGCTCCTTCCTCTCGAAGGAATCAGTTATCCAGA |
| | | TGGATATCATTGATGGATAAAAGCTGGGAGCATTC |
| | | AAATCTTTGACACACAGGGCAACTTCCATCTCGTC |
| | | TTTGGCACTGAGATTTAGAGCATCTTTTGAGTCGA |
| | | ATATACAGTAAAACTGAAAGAGGAAGCAGGGCTG |
| | | TATCTCAAAATACTTTAATCAACTCATCACAACTTT |
| | | CGCAAACAAAGCAAATGCCTGTTATTCTAGATGAA |
| | | GAATCCTATATGAAAGAACCTGGGCTACGGCTGCG |
| | | TAACAAGCCAGATCAAATAGTCAAAAGAGCCTGG |
| | | GAGAGGGCCTAGTAACAAGCCGGATCAAATATTG |
| | | ACACTTGCCCAGGCTGACAGAGTTTCAGGAGTACA |
| | | AGGCCTAAATCGGACTAATACTACGGCCATGGCTG |
| | | AAAGCAATGATTAACTAGAAAGGGACCTCAATCT |
| | | GAATAGCAAATGGAGAAGTAAGCCAATCCTAAAT |
| | | AGAAACATGCTCCTTAATTTTGAAGGGAGAATTCA |
| | | AAACCTAGCAAGTTCAAAATTTATCATGTACACCA |
| | | ATCTCATTAGGCTTATTGTACAGGAAATTTCGTTTG |
| | | ACTAATTCCCACCACTCAAAGAAGGGAACAGCCA |
| | | GCCATCAATGAATTGACTAATAATAAGTGGTAACA |
| | | ATAGATGGACTCATAGAGTACTAGGAATACAAGA |
| | | CAACAAAGACCTAAGACCATTTTAAGCTGTACGCA |
| | | AACAGTGATGCAAGAAAAAGCCTCTCCTATGTGTG |
| | | CATATAAGTTGATGGGTAACTACAATTTTGAAAAG |
| | | ATGGACTCTCACGAAGGTATAGTGGAAGGAAGCA |
| | | ACATAGAATATAATATTATACTGCATTTCTTCACA |
| | | GTAGCAATCATTATGTATACTGGCTCACCTTATTTA |
| | | CAATAATCATTTTACTTTTCCTGTATTTTCATACCA |
| | | AAATTCATTGATAACTCCAAAAATGAACTATTTGA |
| | | CTACATAGATATGACAACCTATGTTACCCGGACTC |
| | | GGGTATGGGTGTCGGACACGGGTTCATATCCAAGT |
| | | GTCGGACTCGGCAATATTATGAAAAATTTACATGT |
| | | TTTTTGCCTAAAATAAGTGTCCAAGTGTCTATACCC |
| | | ATGTCCGAGTGTCGAGTGTCCGACACGGGTACTCG |
| | | AAGCAAAATGAAGAGTCCGGGTAACTAAGATGAC |
| | | AACTGAGGCTCTACTAAAGGCCTAGAAATCAGCTA |
| | | TAAACAGTATTTAGGTATATTTCGAATTACACAGG |
| | | GTTGACTTTAAAATTAATGTTTATTTTGTAAAATAT |
| | | ATAAATGTTAGCTCTCTCTCGACTTGCTAGTATCTT |
| | | TAATCCTTCTAAACTATAACCGTCAATTAGTAAAG |
| | | GTGCATCAAGATTCAAAATACTACACCTAGAAACA |
| | | CAAGGGTAAACACAAAATTCTGAAGATTCACATTC |
| | | TTTGACTGTTTTTATTGATAGCAATAGCCGATGATC |
| | | TGCTGGGCGCCTAATGACAGCAAGATAAAATGCA |
| | | AATACACTAAAAAGAGCAGATATAAAGTACACAG |
| | | CTTCATATTGTTGCAAGACCACCGAGAGTCAAAAT |
| | | ACACATTCACCCCATCAAATCTCAGACTTAAAAAA |
| | | ACAATGAACTTGTAAAACTAGCATATATGATATGT |
| | | TAATTTCTATCGTACACAACACACGGAGTAAAAGG |
| | | GCAATTAAAATTAGCTTTTTAGTTGAAAATATAAT |
| | | TAAAAAATCAGCCACAAGCAAGCAACAAGTTATC |
| | | ATCATACCTGTAGAATTCTTTAATAGTTTCCAATGA |
| | | CTTTTTTCGCAATTGTTCTTCCGGCAACACCTTTTC |
| | | CGTAGAAATGTTTCCTACAGAAATAGACTCCATAC |
| | | CTCTGGAAGGAGGCGTATTAGGCCGAGATCTGTTA |
| | | AAATACGATCTGGATTCTGCATTTCCCTGTTCATAT |
| | | AAGTCGAATTTCCATCCGTTATATTCATGTGGTCAT |
| | | GAGCAGACGGGCTGACAAATCTCTGAGCAGATCG |
| | | CGGAATCAGTTCCTCTCTTGAGGTATAAACTGGCC |
| | | GGTCTGTTACAGAACCGTACCCATTGAGACCAGGG |
| | | GTAGTCCTTCTGGAATCTTGATTGTGCATGTCAGA |
| | | GAAGGGTACGCTTAACATTGGGGGCTGTCCTCTAA |
| | | TAGACATTCCCCTGGCAAGACCACCTTGGGGCACA |
| | | AGGGTAATGGAGTCACCAAGGGGTCTTCTGTGGCA |
| | | ATAGAGAATTCCTGTTATCAAAAGGGTGTCTCTCA |
| | | CTGTCCTAACTATCTTGAGTGGCATAACCGCGTGG |
| | | TTGTTGTGGTGCTCCACGGAATCCTCCCATATGGG |
| | | AATTTGGCGATGATAATACATTTGACCCTCTTGGA |
| | | GCAAAGTCCATGGGTTGTTGACCCCTTCTAAAAGA |
| | | TGAACCCATACTCGGTCCACGAGCTGAATCTGTTA |
| | | GCTTGTGCCTGCCTCTCATTCGCAGCATCCCTGTGC |
| | | ACTTCCTCGATCTTTTTTGGACCTTCAACTTTCCTC |
| | | CTCTGCTGCCACTTGTTTTTCCTGCAGGTCCATTGA |
| | | ATCCTTCAGCATGAACCTCACTCTCGAAGAGAGCT |
| | | TCATGTTATTAGATAGCTTGGCCATCATATCAAAA |
| | | TATGCATCCATGTGTTCCTTGGCTTTAGGGTGATCT |
| | | ATCATCTCTCCTATTGTGCTCATTAGTTTGCACAAG |
| | | GCTTCTACATCTTCCTCATCAGGATTTTGATACTGG |
| | | CCCAATAGTTTCTTAATGCATTCATGCATGATCCTC |
| | | TCTGTTAACATCTTTTTCTTGCATAATTCCCCAATA |
| | | AGTCTTGATGTTGCCCAACATTCGCCTTCGTGCCTG |
| | | GACTCTCTTCCTTCCCTTTCTTCATCAGATTGTTTA |
| | | ACCTCACCTTCCTCTTCAGCTCTGTTGGCTTCTTCT |
| | | TGTTCCCTCTCACCTCTCTCAAACTCCTCCTGGCAC |
| | | TTATTCAGAAAGTAGCCTCTTAAATGTAATTTTTTC |
| | | GTTGTCTTCATCAAAATCAGGCAAATCGCCAGCAA |
| | | GATGATAGCAGAAATCAGCATACATCTCACAAAAT |
| | | GTCGGTTCCATAAGAGCTTTATCAAAGATTTGAGA |
| | | TATCACACCAGTGAGAGTACCAGCATTGTCAATGT |
| | | TAACTTGTCTTGACTTGCTCAAATAGTCTTTCAAAG |
| | | TTTTGCGGTGTTAACTTATTCAGAATTGCTTTCAAC |
| | | TGCCTTTGCTTTAGCCTGTTCCTCGTCAGTCATTTT |
| | | ACCCACTTCATACTTTCTCTAGCCTTATGCATCACC |
| | | TGTGCGGGAGTCCGAGGAGAAGGCATTAGACCTCT |
| | | CTGAAAATTAGTCGCCCGCTGCCATTTGTCGGCAT |
| | | CAGAGTCAGCTTCGCTGCATTTCTATACTGAGGAC |
| | | CCATAGAATGAACTGGCCCAGATAGGATTCCACCG |
| | | GAATACATAACTGGTGATTGAACACGTGGATTTCT |
| | | CAATACTCCAAAATTTGCTCCAGGTCGAAAAACAT |
| | | TGTTTGCATGACTTCCATAACCCATATCCAGTACA |
| | | GGGGAAGAGGCCCGGGTACCTTACTCCATTTATCA |
| | | TCATCGCCCATACCCATGCCAACACCACGGCGACC |
| | | TGATCCAGACCTTCCCATTGACCTATCGATTATCTC |
| | | TTCCTGGGACTGGGCAAGGAACATTGCCAGTAGAC |
| | | AATACCCAATGCTTCTGCAATATCTGGGGTGATCT |
| | | CAAAGCCCTCAGGAAGATCCTTGCATTGCTCTGAA |
| | | AATTTAAGAAGGAAATCTCTAGAGTACTTTCTGTC |
| | | CATTAAAGTTTTTGTCCTCAATGTGATCTTTTACCT |
| | | CTCCAAGGAGCTTCCCATCATCAAGTTTTGGAGTT |
| | | GATAAATCTGCAGCATCTTCCCAAGTCATCTGGTT |
| | | CAGCTTTACTCTCATTTGGAACATCTTTCGGAGAA |
| | | ATTTAGAAGCCAACTCTTTAGCTTGGCTAGAAGAT |
| | | GTTTCTGAGCTCTCAACATCGATCGTGATTTTCTTT |
| | | CTTTTCATCTGGACCTTTATATGCCATATAAAGATC |
| | | AGCAGTTGTACCAGCACGGTTTGCCTTTTGTAAAG |
| | | CTTCTTTTCTCTTCTTCTTTGTATTTGTATTCTTTGA |
| | | CATAATTGTCTCCGGCACAGGTTTATCTTTCAAATC |
| | | AATTGATGAAGAAAGCAATTCTGTGCTATCACTAT |
| | | CAGCAATTTCAGTTTCACAAGGCGGCTCAGACTGA |
| | | GATGGAACTGAAAGAATTTGTGATTTCTGATCTAA |
| | | CTTGTCGGATTCTGTTTCTGTACTAGCCATGGTATT |
| | | ATCGATTATAGTAGAAGCATCCGAAGCTGAAGTCT |
| | | TATCATAATCATGTGAGATTGTGGATCCAAGAGAC |
| | | AATGAGGATGTTTCAGTAATCAGGCAGTCCACTGT |
| | | CCCAACTATAGAGGTTGATAAATCAGCATCTGCTT |
| | | CACCTTTTTGTGAGGTGTACAGGTTCCATCTCCAAC |
| | | TTCAGCTGTTTATCTTTCACTTCCATGATAAGGCTC |
| | | TCAGTTTGATCAGCATGTTCATATGGTCTTGGTGAT |
| | | ATCGGATTTTGCACTTGCCACACAACCAGCAGCAT |
| | | CTGCATTTGGCTCAGAGAGCAGTATGTTTCTACTTT |
| | | CTTGAGTTCGCATAGCTTCAATGTTTTGGCTTATTA |
| | | TCCACAGATACATCAGCTGACATTTCAGCAGAATG |
| | | ACTCCATCGCTTTTTTCCAACTCATGAATTCCCTGT |
| | | ACCTCAGAACTATTTGTATTTAAGTCAACGGCAAC |
| | | AGAGGTTTCTGATTCCCTGACATTTTCCTCTCCTGG |
| | | CTGAGCTTCTGGTATAGAAGATGATCTTCTGTCCTC |
| | | TACAACTTCGGGTAACGAAGGGGCAACAGGTCGA |
| | | TCAATCTGAGAACAAAATTTGTTAAAACCATACAC |
| | | TACAGCAACTGAAGTATACTGCTGGAATAAAAATC |
| | | TTAAAAGAGGGTAATACCTGATTCTGCAGTACAGA |
| | | TAGGCTCTCTTACTGTGTTTCTCATCATTATCATTA |
| | | GACCTATCCAATAGATCATTTTTTGCACCTACAGT |
| | | AATACTAGAGCCTGTGGAATACTCGGAATTTTCAG |
| | | GAGGTGTACTAGGAATAGAACTTGGTAGAGGGGC |
| | | TAGATGCGGAAGATGAATCAGATACTCCGCCAGTT |
| | | GCTTTAATTTGAACAGGTATCGATGTATACTCTGA |
| | | CTTCTGTGAGGACATATCACTTGTCTTTCCAGGATC |
| | | TCTTTCAGTACAAATAGATGTAGCTTCTCCTCTACC |
| | | ATATACTTGAGGAATATTTTTCTCAACAGTAGGTG |
| | | AGCTTGCTGGCAAAGAAGTTTCAGCAATTTTTTCC |
| | | AAGCGTGGGAGCTGCTGGCTTCACGGTGACCTGTA |
| | | TTTTAAATGAGCATGTACCGTAAATAACATTTCAG |
| | | TAATGGCATGCAACAAAATACCAGCAGTGGATAA |
| | | AACAACACGGTAATACGGTTTAATAAAGACAGGC |
| | | TAACCTGTTTATATAACCTTGGAGCCTGTGAATTTG |
| | | GAGGAACTTGGTTACTGCTTAAATGAAGAGAATTA |
| | | GGACCTGTATAAAATACGGAACCCTGACTGTAAGA |
| | | ATTAGGGTAAAAGTTACTTGGACGGCCAGTAGGA |
| | | AAAGATGAAATAGGTTGGGACTGAGGTGGTATATT |
| | | GGATGTGATCGCGGAGCAGATCGAACCAGATTCTT |
| | | TGTAAGTATCAGTCTTTTTAGATAAACTCAACTCTT |
| | | CATGAGTATCAGGGTGAGTTATCTTAACTGTTTTA |
| | | CGAGCACCACCAAAATTTCCTGGCTGCTGCTGCGA |
| | | GAACTGGGGATTGAGTGTTCATCCCCATATTCCCC |
| | | ATGTGAGGTAACTGGGTACCCATTTGCGAGGAGAA |
| | | ATTGACACCCTGACTCTGATGCATGACACCCTGAG |
| | | GTGGCATCATGTGGGGTGGAAGACCCTGAATATAC |
| | | AGTTGCTGCTGCACTTGAGAAGGATTTCCCATAGG |
| | | GAAGGGCATTCCCATTGGCATTGGCATAGAGCTAT |
| | | TTACCATGCTTTGGGACTGCAACTGTGGATTAGGA |
| | | TTCCCAAATTGAACTGGTATATGTTGCTGGTGAAA |
| | | TGGCATTTGCATAGGCATCCCAGAAATAGGGTGAA |
| | | CAGAAGGCTTCTGAGTTTGGATTGCATGTGGACCA |
| | | AGGTGCAACTTGCACATCTCTTGCTTTTGACATCG |
| | | AATGAGTGTCAACAATATTAGGTTGGTCAGAAGTT |
| | | GCAGAATTATTGGTAGGCACATTCTGCTTAGGAAC |
| | | GGATTGGGCTGGCAGCACAGGCATAACTCTTGATG |
| | | ATTCATAGCGAGCCTGCAAAAATAATTGACAAGCA |
| | | GGAAAGGAAGTTGTCAAACAATTAATATCAGCAG |
| | | GGATAAAGAGCAGAGCATAATCAGCCTATTCTGTA |
| | | ATTTAATCAACTCATCTCATTGCACTAGGGCCAAA |
| | | AGTCATTCTACAGAAAGCACATCATATTACAATTA |
| | | ACCAAACCCTTTTTCAAGCTCTATAATAGATTACTC |
| | | AAAAACTGAGATTGCAATTTCAAGTGTGTCGTTCG |
| | | AAAAAATAGAGATGCAACAAATGTGGGACACCAC |
| | | AAAATGTAGCCTATCCATTTGGACCAGAAGGGGG |
| | | ATCATAAAGTAATATACTTCACATCAACCATTTAG |
| | | AAATCAAAGTTACAAATGATCTAGCCTCCCTATTA |
| | | TAATAAAGAGAATACTGATATTAAAAATATTCATC |
| | | TAGAAATTGCAAGTAAATCCTATTTGCAAGCCCAC |
| | | CCCCACATCACAATGAACAAAAAACATGGCAACA |
| | | ATGTTTAAAACCTGTTCCCGTTTCTGGCTCATCCAA |
| | | ATTTGGCGGCGCTGAGCTAGTTTCGAGCAGGTACC |
| | | TAAACCACAATCCGAAATCATTCCAGTAATTATAA |
| | | TTATATGAAGACCAATATCATTCTTAAGCTTCAGG |
| | | TAACTGAGATAGCTAACAATATACATTCTTGATGA |
| | | AAACTTTCAGCCCTACCTGCATCAGACTAGGACTC |
| | | TATTGATCAAACCTGGAGAGGAAAACCTCCACCTG |
| | | AGCCACGACCACAAAACTCCAATTAGCTTATTATG |
| | | AATACAGAAGTTCATATATTTAACTTCATCATGTA |
| | | CAATGCCAAATATTTACATAGAACGACACTTAACC |
| | | TTTAGCAGGGGTTGAAGGGGCCATTGTATTTGAGC |
| | | TTACGGGGTATTATTTGGAGCAGGAGCTTTCGGCA |
| | | TATCCCCGATTAATTTTTGGTGCCGGAGTAACATTC |
| | | TGCTTGACAACAGCTGGAGCATCAGACCCTCCTAC |
| | | AACAAGGACAACAAAATAAAAACCATACTCGACC |
| | | ATCACACCACATTCAAAAACAAAAACAATTAAAA |
| | | TCCTTGAACAAAATCTCTTTCAATACCATGTGATTG |
| | | TGAATGAGCGCCATTCTTGCACACCACGGGCTCAA |
| | | CCAGAAACAGCAACATTACCACCGGCAGTAGCAT |
| | | CATTCCTAATATCCGAATTCACAACCCGAATTTGC |
| | | GATCCTTGCGCATTACTAGGCTTCTTAAAACTACA |
| | | AAAAATAAAAAAAAATTATTAGAATCATCAGAAA |
| | | AGTACACAAAACAGACAAGTATACTCTATAACTTC |
| | | ATACTGTCCGGACCAAAGATTCTATTACTTAATTA |
| | | ATAAAGCTTACAAATTAACTACAGGTAAAACAAA |
| | | CCTAAGTATCCTTGATAAATTAAAAAAAACACATT |
| | | CTTTAATCTTAGTATATAACCAATAATCACGTATCT |
| | | AATTTATCCAATCAAGCACTAAGACAATCTTAGGT |
| | | CACGTCAATCACCCCCACATATTAATTTATGAAGA |
| | | CTAAAAACCAATCAAGCACTTATTAGCTAACTAAT |
| | | CAATCACGTAGTATTTAATTATCGAGATTATGTAA |
| | | CCAACCAAGTACCAACAATCTTAGGTTACTAACCA |
| | | ATAGCGTATTATATTGTCAAGATTACTAACCGAGT |
| | | ACCAACAATCTTAGGTCACTAACCAATTGCGTATC |
| | | TAATTATTCAAATTACTAACCAACCCAGTACCAAT |
| | | TAATCTTAGACAACTAACAAATTGCATAATAATTT |
| | | ATCGAGGTCTAAATGTACACACAATTACAAAAAAC |
| | | AAAGACAGGAAACCCAATATACCTTCGACTAGTCG |
| | | AAAGCGAAGGTAGAGGGGCGGTGGCAGCACCGCT |
| | | ACCCTTACCACCACCGCCTGAAAAATTCCTCTGCC |
| | | CACCGGAGCTATCACCCGGATCGATTAGTTTTTCG |
| | | ATACTGTCCCGACTCGCTCTTCTGAGCCCTAGATTG |
| | | ATTGACGGACATAAATCCAAACCCTAATCACCTCC |
| | | AATCTTAATCAAATCAACAATTTTCTGCACCAAAA |
| | | ACACAGATCTAATTCATTAATCACATAATCAACAA |
| | | AAATTCAAAACACATTACAATCAATTCATACACAC |
| | | AAACATATATACATGTAATTAAAGATGCTTAATTG |
| | | ATTAAAGATTGAATCTTTTTGGTGAAATTGAGTGA |
| | | ATAGAGTGAAGATATATGTATAATAATTGTGTAGG |
| | | CGTGTAAGAATTGTGTGTATATGTGTATGTATAGA |
| | | TTGGATTGAATTGGATGGAAGGGGGTGAAGAAGA |
| | | GGAAGACAGAGAAGGAAAAACTGAACCCAAGTGT |
| | | ATGTGTGTGTTTGTGTGAATTGTGTGTGTGAGAAA |
| | | AGAAGTGTGTTTTAACTTTTATATGTGTATATATAA |
| | | AATCAAGAATTAAAGTATCATTGTTTTCAAATTGG |
| | | AATTCAGAAAAAATTAAATTAAAATAATATAATTG |
| | | TTGTTTAAAAGCTTCCTTTTGAGAAAATTACAAATT |
| | | TTAGATTATATATGGACGATGGTCAAAAATATTAT |
| | | TTTTTAATATAATATGACAAACATACTATTTGTAA |
| | | AATTGATGGTCAAAAATATCAACCAAGTACATATT |
| | | TCGAAGAATTGATAATCTAATACGTATTTGATGAG |
| | | CTACGGAGTTTTACCCGCGATACGCAAGATTGTTA |
| | | TCCGAAATTTGTTAAACGTAAATTGTTATATAAAT |
| | | GAAAAAAAATCGGTATATCGTATTCCGAGATGATG |
| | | AACTCTTAATTATATTAACGAACAAATAAAAATAC |
| | | ATAAAGATGCACCAAAAAATATAAATCGTCACTTT |
| | | CAAAGAAAAATACAGGAACCTATTGTTTGATTAAA |
| | | ATCTCACATCAATGTCAGGATTATATTCGCCACAA |
| | | AGATTTCAGCGTTAAGCAATTGACGATTATAAGAA |
| | | GTGAACATAGTAACAATCTGAATGCTTTGTTAATT |
| | | AATATAACGCGTACATTACTTAACGGAAACGTAAA |
| | | TGTGTACCAGTGTGGCCAGATAGACTATTCACCTT |
| | | CTTATAATTACACTTTACTACATTAGTCTTTTGCAT |
| | | GACACTATTTTTCTTGGGAGATGCAATATATAAAA |
| | | TGCTTTAACTCTAATGTCCCCACAAATGTCATGTCG |
| | | TGTTTAACAATACATGTTTGTTTAATTGTACAAACA |
| | | GTTAAATCTCGATATTATAATACTTTATAACTTCTG |
| | | AAATATGGGTTAATTATATCTAACTTCGGTGAATC |
| | | TAAAATTAAGTAATATGGTTTAATATCATATATGT |
| | | TAGCATAAAATATGTTAGTATAAAATATAAAGTTT |
| | | GTCATACAACGATAACATTTTCTTTTAATTTAATAT |
| | | TAGTAACAAATTATCCCTTTAATATCATTCTAAGC |
| | | AATAATATAAACAAACTAATGATAACTGATCTTCT |
| | | ATATAATT |
| 11 | cDNA, | >CDNA_RES |
| | resistant | ATGTATATATGGCTCAGAAGAGCGAGTCGGGACA |
| | | GTATCGAAAAACTAATCGATCCGGGTGATAGCTCC |
| | | GGTGGGCAGA |
| | | GGAATTTTTCAGGCGGTGGTGGTAAGGGTAGCGGT |
| | | GCTGCCACCGCCCCTCTACCTTCGCTTTCGACTAGT |
| | | CGAAGTTTTAAGAAGCCTAGTAATGCG |
| | | CAAGGATCGCAAATTCGGGTTGTGAATTCGGATAT |
| | | TAGGAATGATGCTACTGCCGGTGGTAATGTTGCTG |
| | | TTTCTGGAGGGTCTGATGCTCCAGCTGT |
| | | TGTCAAGCAGAATGTGGAGGTTTTCCTCTCCAGGT |
| | | ACCTGCTCGAAACTAGCTCAGCGCCGCCAAATTTG |
| | | GATGAGCCAGAAACGGGAACAGAGATGT |
| | | GCAAGTTGCACCTTGGTCCACATGCAATCCAAACT |
| | | CAGAAGCCTTCTGTTCACCCTATTTCTGGGATGCCT |
| | | ATGCAAATGCCATTTCACCAGCAACAT |
| | | ATACCAGTTCAATTTGGGAATCCTAATCCACAGTT |
| | | GCAGTCCCAAAGCATGGTAAATAGCTCTATGCCAA |
| | | TGCCAATGGGAATGCCCTTCCCTATGGG |
| | | AAATCCTTCTCAAGTGCAGCAGCAACTGTATATTC |
| | | AGGGTCTTCCACCCCACATGATGCCACCTCAGGGT |
| | | GTCATGCATCAGAGTCAGGGTTTCTCGC |
| | | AGCAGCAGCCAGGAAATTTTGGTGGTGCTCGTAAA |
| | | ACAGTTAAGATAACTCACCCTGATACTCATGAAGA |
| | | GTTGAGTTTATCTAAAAAGACTGATACT |
| | | TACAAAGAATCTGGTTCGATCTGCTCCGCGATCAC |
| | | ATCCAATATACCACCTCAGTCCCAACCTATTTCATC |
| | | TTTTCCTACTGGCCGTCCAAGTCACCG |
| | | TGAAGCCAGCAGCTCCCACGCTTGGAAAAAATTGC |
| | | TGAAACTTCTTTGCCAGCAAGCTCACCTACTGTTG |
| | | AGAAAAATATTCCTCAAGTATATGGTAG |
| | | AGGAGAAGCTACATCTATTTGTACTGAAAGAGATC |
| | | CTGGAAAGACAAGTGATATGTCCTCACAGAAGTCA |
| | | GAGTATACATCGATACCTGTTCAAATTA |
| | | AAGCAACTGGCGGAGTATCTGATTCATCTTCCGCA |
| | | TCTAGCCCCTCTACCAAGTTCTATTCCTAGTACACC |
| | | TCCTGAAAATTCCGAGTATTCCACAGG |
| | | CTCTAGTATTACTGTAGGTGCAAAAAATGATCTAT |
| | | TGGATAGGTCTAATGATAATGATGAGAAACACAGT |
| | | AAGAGAGCCTATCTGTACTGCAGAATCA |
| | | GGTATTACCCTCTTTTAAGATTTTTATTCCAGCAGT |
| | | ATACTTCAGTTGCTGTAGTGTATGGTTTTAACAAAT |
| | | TTTGTTCTCAGATTGATCGACCTGTT |
| | | GCCCCTTCGTTACCCGAAGTTGTAGAGGACAGAAG |
| | | ATCATCTTCTATACCAGAAGCTCAGCCAGGAGAGG |
| | | AAAATGTCAGGGAATCAGAAACCTCTGT |
| | | TGCCGTTGACTTAAATACAAATAGTTCTGAGGTAC |
| | | AGGGAATTCATGAGTTGGAAAAAAGCGATGGAGT |
| | | CATTCTGCTGAAATGTCAGCTGATGTATC |
| | | TGTGGATAATAAGCCAAAACATTGAAGCTATGCGA |
| | | ACTCAAGAAAGTAGAAACATACTGCTCTCTGAGCC |
| | | AAATGCAGATGCTGCTGGTGAAGCAGAT |
| | | GCTGATTTATCAACCTCTATAGTTGGGACAGTGGA |
| | | CTGCCTGATTACTGAAACATCCTCATTGTCTCTTGG |
| | | ATCCACAATCTCACATGATTATGATAA |
| | | GACTTCAGCTTCGGATGCTTCTACTATAATCGATA |
| | | ATACCATGGCTAGTACAGAAACAGAATCCGACAA |
| | | GTTAGATCAGAAATCACAAATTCTTTCAG |
| | | TTCCATCTCAGTCTGAGCCGCCTTGTGAAACTGAA |
| | | ATTGCTGATAGTGATAGCACAGAATTGCTTTCTTC |
| | | ATCAATTGATTTGAAAGATAAACCTGTG |
| | | CCGGAGACAATTATGTCAAAGAATACAAATACAA |
| | | AGAAGAAGAGAAAAGAAGCTTTACAAAAGGCAAA |
| | | CCGTGCTGAAACATCTTCTAGCCAAGCTAA |
| | | AGAGTTGGCTTCTAAATTTCTCCGAAAGATGTTCC |
| | | AAATGAGAGTAAAGCTGAACCAGATGACTTGGGA |
| | | AGATGCTGCAGATTTATCAACTCCAAAAC |
| | | TTGATGATGGGAAGCTCCTTGGAGAGTCCCAGGAA |
| | | GAGATAATCGATAGGTCAATGGGAAGGTCTGGAT |
| | | CAGGTCGCCGTGGTGTTGGCATGGGTATG |
| | | GGCGATGATGATAAATGGAGTAAGCGGGCGACTA |
| | | ATTTTCAGAGAGGTCTAATGCCTTCTCCTCGGACTC |
| | | CCGCACAGGTGATGCATAAGGCTAGAGA |
| | | AAACTATTTGAGCAAGTCAAGACAAGTTAACATTG |
| | | ACAATGCTGGTACTCTCACTGGTGTGATATCTCAA |
| | | ATCTTTGATAAAGCTCTTATGGAACCGA |
| | | CATTTTGTGAGATAGGCTACTTTCTGAATAAGTGC |
| | | CAGGAGGAGTTTGAGAGAGGTGAGAGGGAACAAG |
| | | AAGAAGCCAACAGAGCTGAAGAGGAAGGT |
| | | GAGGTTAAACAATCTGATGAAGAAAGGGAAGGAA |
| | | GAGAGTCCAGGCACGAAGGCGAATGTTGGGCAAC |
| | | ATCAAGACTTATTGGGGAATTATGCAAGAA |
| | | AAAGATGTTAACAGAGAGGATCATGCATGAATGC |
| | | ATTAAGAAACTATTGGGCCAGTATCAAAATCCTGA |
| | | TGAGGAAGATGTAGAAGCCTTGTGCAAAC |
| | | TAATGAGCACAATAGGAGAGATGATAGATCACCC |
| | | TAAAGCCAAGGAACACATGGATGCATATTTTGATA |
| | | TGATGGCCAAGCTATCTAATAACATGAAG |
| | | CTCTCTTCGAGAGTGAGGTTCATGCTGAAGGATTC |
| | | AATGGACCTGCAGGAAAAACAAGTGGCAGCAGAG |
| | | GAGGAAAGGGTCAACAACCCATGGACTTT |
| | | GCTCCAAGAGGGTCAAATGTATTATCATCGCCAAA |
| | | TTCCCATATGGGAGGATTCCGTGGAGCACCACAAC |
| | | AACCACGCGGTTATGCCACTCAAGATAG |
| | | TTAGGACAGTGAGAGACACCCTTTTGATAACAGGA |
| | | ATTCTCTATTGCCACAGAAGACCCCTTGGTGACTC |
| | | CATTACCCTTGTGCCCCAAGGTGGTCTT |
| | | GCCAGGGGAATGTCTATTAGAGGACAGCCCCCAAT |
| | | GTTAAGCGTACCCTTCTCTGACATGCACAATCAAG |
| | | ATTCCAGAAGGACTACCCCTGGTCTCAA |
| | | TGGGTACGGTTCTGTAACAGACCGGCCAGTTTATA |
| | | CCTCAAGAGAGGAACTGATTCCGCGATCTGCTCAG |
| | | AGATTTGTCAGCCCGTCTGCTCATGACC |
| | | ACATGAATATAACGGATGGAAATTCGACTTATATG |
| | | AACAGGGAAATGCAGAATCCAGATCGTATTTTAAC |
| | | AGATCTCGGCCTAATACGCCTCCTTCCA |
| | | GAGCCCTGCTTCCTCTTTCAGTTTTACTGTATATTC |
| | | GACTCAAAAGATGCTCTAAATCTCAGTGCCAAAGA |
| | | CGAGATGGAAGTTGCCCTGTGTGTCAA |
| | | AGATTTGAATGCTCCCAGCTTTTATCCATCAATGAT |
| | | ATCCATCTGGATAACTGATTCCTTCGAGAGGAAGG |
| | | AGCATGGAGAGGGGTTTGAATCTGTTC |
| | | TAGCTTGTTTGGAGGATGCTGTAACTGATGCTCCT |
| | | AAAGCACCGAAATTCTTGGTGCTTGTAGAAATAGG |
| | | GCTTGCAGCTGAAGTTCTGGAAGCGTCC |
| | | TGGAGATTATTAAAACTGAAAAAGGAGAACAG |
| Seq | Protein, | >Protein_Res |
| IND no | Resistant | MYIWLRRASRDSIEKLIDPGDSSGGQRNFSGGGGKGSGAA |
| 12 | | TAPLPSLSTSRSFKKPSNAQGSQIRVVNSDIRNDATAG |
| | | GNVAVSGGSDAPAVVKQNVEVFLSRYLLETSSAPPNLDEP |
| | | ETGTEMCKLHLGPHAIQTQKPSVHPISGMPMQMPFHQQHI |
| | | PVQFGNPNPQLQSQSMVN |
| | | SSMPMPMGMPFPMGNPSQVQQQLYIQGLPPHMMPPQGV |
| | | MHQSQGFSQQQPGNFGGARKTVKITHPDTHEELSLSKKTD |
| | | TYKESGSICSAITSNIPPQS |
| | | QPISSFPTGRPSHREASSSHAWKKLLKLLCQQAHLLLRKIF |
| | | LKYMVEEKLHLFVLKEILERQVICPHRSQSIHRYLFKLKQL |
| | | AEYLIHLPHLAPLPSS |
| | | IPSTPPENSEYSTGSSITVGAKNDLLDRSNDNDEKHSKRAY |
| | | LYCRIRYYPLLRFLFQQYTSVAVVYGFNKFCSQIDRPVAPS |
| | | LPEVVEDRRSSSIPEA |
| | | QPGEENVRESETSVAVDLNTNSSEVQGIHELEKSDGVILLK |
| | | CQLMYLWIISQNIEAMRTQESRNILLSEPNADAAGEADAD |
| | | LSTSIVGTVDCLITETS |
| | | SLSLGSTISHDYDKTSASDASTIIDNTMASTETESDKLDQKS |
| | | QILSVPSQSEPPCETEIADSDSTELLSSSIDLKDKPVPETIMS |
| | | KNTNTKKKRKEAL |
| | | QKANRAETSSSQAKELASKFLRKMFQMRVKLNQMTWED |
| | | AADLSTPKLDDGKLLGESQEEIIDRSMGRSGSGRRGVGMG |
| | | MGDDDKWSKRATNFQRGLMP |
| | | SPRTPAQVMHKARENYLSKSRQVNIDNAGTLTGVISQIFD |
| | | KALMEPTFCEIGYFLNKCQEEFERGEREQEEANRAEEEGE |
| | | VKQSDEEREGRESRHEGE |
| | | CWATSRLIGELCKKKMLTERIMHECIKKLLGQYQNPDEED |
| | | VEALCKLMSTIGEMIDHPKAKEHMDAYFDMMAKLSNNM |
| | | KLSSRVRFMLKDSMDLQEKQ |
| | | VAAEEERVNNPWTLLQEGQMYYHRQIPIWEDSVEHHNNH |
| | | AVMPLKIVRTVRDTLLITGILYCHRRPLGDSITLVPQGGLA |
| | | RGMSIRGQPPMLSVPFSD |
| | | MHNQDSRRTTPGLNGYGSVTDRPVYTSREELIPRSAQRFV |
| | | SPSAHDHMNITDGNSTYMNREMQNPDRILTDLGLIRLLPEP |
| | | CFLFQFYCIFDSKDALN |
| | | LSAKDEMEVALCVKDLNAPSFYPSMISIWITDSFERKEHGE |
| | | GFESVLACLEDAVTDAPKAPKFLVLVEIGLAAEVLEASWR |
| | | LLKLKKEN |

Comparison of domain predictions (InterPro) of the functional protein originating from the susceptible plants with the non (fully-) functional protein originating from the resistant revealed that the latter is missing the Med15 domain. It can be speculated that in the resistant plants, wherein the protein is lacking the Med15 domain, the virus cannot hijack the protein.

### Example 6. Virus Induced Gene Silencing Experiment (VIGS) to Silence the Genetically Encoded Resistance from Apium graveolens

Tobacco rattle virus (TRV)-derived VIGS vectors have been abundantly described to study gene function in Arabidopsis thaliana, Nicotiana benthamiana, Lycopersicon esculentum and other plants. Prior to the experiment we received plasmids 0155-157 pTRV1 and 0158-160 pTRV2-MCS from the Arabidopsis Biological Resource Center.

To confirm that eIF4G is involved in the mechanism of resistance against CeMV, eIF4G (as referred to in Seq ID No 7 and Seq ID No 8) can be silenced with VIGS.

For this purpose a VIGS constructs targeting eIF4G (Seq ID No 8) in susceptible plants were designed with siFi21. In addition, a positive VIGS control was used, and this positive control targets the (Phytoene Desaturase) PDS gene. The negative control is the empty vector.

Susceptible celery plants are targeted with either a VIGS1, PDS1 (positive control) or an empty plasmid (negative control); Plants are subsequently infected with CeMV (see example 1). Plants wherein the VIGS silencing was successful for VIGS1 construct will become resistant to CeMV as can be confirmed by visual inspection (see example 1).

| Seq | | |
|---|---|---|
| ID No. | Type | Sequence |
| 13 | VIGS target | TCAATTTGGACATGTTTCTTCTTAATAATAATTTTAATAATAATAAT |
| | PDS | TTCAAAAATGTTCAGAACCTGTTTGG |
| | | GAACTAGGAATTGACAACCGATTGATTTTCCGGAAGTTCTACCTCA |
| | | GGATCATGAGTCGCTGCTTTCTCGCAGGAAGCATGTGCTGG |
| 14 | VIGS target | GGTAATGTTGCTGTTTCTGAATCCTTCTCAAGTGCAGCAACTCATG |
| | elF4G gene | AAGAGTTGAGTTAAAATATTCCTCAAGTATATCAAGTGATATGTCC |
| | | TCACAGCACCTCCTGAAAATTCCGAGTATTCCATTTAACAAATTTT |
| | | GTTCTCAGATTGATCGACCTGTTGCCCCTTCGTTACCCGAAGTTGT |
| | | ACCAAATGCAGATGCTGCTGGTGAAGCAGATGCTGATTTAAAGAA |
| | | TACAAATACAAAGAAGAAGAGAAAAGAAGTCAACTCCAAAACTTG |
| | | ATGTTGAGAGAGGTGAGAGGGAGAAGAGGAAGGTGAGGTTAAAA |
| | | GAAAGGGAAGGAAGAGAAAGAAAAAGATGTTAACAGATGAATGC |
| | | ATTAAGAAACTATTGGGCCAGTTTGCTCCAAGAGGGTCAACTGTA |
| | | ACAGACCGGCCAGTTCAGTTTTACTGTATATTCGGAAGTTGCCCTG |
| | | TGTGTCAAA |

### Example 7 Crispr-Cas Experiments

To assess whether the wildtype eIF4G is indeed an essential gene involved in CeMV infection of the celery plants, a knockout of the gene can be made by CRISPR/Cas9. A target site was identified and SEQ ID No. 15 can serve as guide RNA (gRNA). The CRISPR machinery, gRNA and Cas9 proteins, can be delivered via either PEG mediated transfection of ribonucleoproteins (RNP), or Agrobacterium-mediated gene transfer. Edited cells will be regenerated to obtain viable plants and PCR or sequencing will be used to confirm the CRISPR event. Wildtype plants and mutants can be infected with CeMV, see example 1 for details, and the hypotheses (wildtype plants, comprising the functional eIF4G protein, will be infected; mutant plants, where eIF4G has been edited, will show no infection). The experiment can be scored phenotypically.

| | Target | Start | Stop | Sequence |
|---|---|---|---|---|
| Seq ID | eIF4G_cDNA | 1382 | 1406 | TTTTCCAAGCGT |
| No. 15 | | | | GGGGAAGCTGCT |

1. A CeMV resistant celery plant comprising a genomic region between 80,545 and 80,589 cM on chromosome 10, said genomic region comprising one or more mutations causing said CeMV resistance.

2. The CeMV resistant celery according to claim 1, wherein said mutated genomic region is 8.8 Mbp between base pair positions 80,246,438 and 89,075,890.

3. The CeMV resistant celery according to claim 1, wherein said mutated genomic region is derived, originates, or is from a celery plant deposited at NCIMB with deposit number 44126.

4. The CeMV resistant celery according to claim 1, wherein said one or more mutations cause the absence of a protein encoded by a cDNA sequence represented by SEQ ID No. 8 in said celery plant.

5. The CeMV resistant celery plant according to claim 1, wherein said one or more mutations are in a genomic region represented by SEQ ID No. 7.

6. The CeMV resistant celery plant according to claim 1, wherein said one or more mutations are deletions, insertions or substitutions in the genomic region represented by SEQ ID No. 7 resulting in the absence of a protein encoded by SEQ ID No. 8 in said resistant celery plant.

7. The CeMV resistant celery plant according to claim 1, wherein said one or more mutations are deletions, insertions or substitutions in the cDNA sequence represented by SEQ ID No. 8 resulting in the absence of a protein encoded by SEQ ID No. 8 in said resistant celery plant.

8. The CeMV resistant celery plant claim 1, wherein said one or more mutations comprise:
a) deletion of at least 4 consecutive amino acids in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
b) amino acid substitutions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
c) single amino acid deletions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus; and/or
d) amino acid insertions in one or more domains of the amino acid sequence of SEQ ID No. 9, wherein the domains are selected from the group consisting of domains at positions 88-103, 120-163, 183-228, 327-350, 485-497, 691-733, 860-886, 940-994, 1022-1092, 1119-1150, 1185-1210, 1562-1580, 1690-1722 and from 1754 to the C-terminus.

9. The CeMV resistant celery plant according to claim 1, wherein said one or more mutations comprise:
a) deletion of at least 4 consecutive amino acids in the amino acid sequence of SEQ ID No. 9 within 20 amino acids of positions 586 and/or 1593; and/or
b) amino acid substitutions in the amino acid sequence of SEQ ID No. 9 within 20 amino acids of positions 586 and/or 1593; and/or
c) single amino acid deletions in the amino acid sequence of SEQ ID No. 9 within 20 amino acids of positions 586 and/or 1593; and/or
d) amino acid insertions in the amino acid sequence of SEQ ID No. 9 within 20 amino acids of positions 586 and/or 1593.

10. The CeMV resistant celery plant according to claim 1 comprising the genomic sequence of SEQ ID No. 10, or sequences having at least 90% sequence identity with SEQ ID No. 10.

11. The CeMV resistant celery plant according to claim 1 comprising a protein comprising the amino acid sequence of SEQ ID No. 12, or sequences having at least 90% sequence identity with Seq ID No. 12.

12. The CeMV resistant celery plant according to claim 1, wherein said genomic region is as defined in any one of the claims 1 to 9 or the genomic sequence of claim 10 is homozygously present in said resistant celery plant.

13-16. (canceled)

17. Seeds or plant parts of the CeMV celery plant according to claim 1.

18. A method for identifying a CeMV resistant celery plant, the method comprising the steps of:
isolating genomic DNA from said celery plant; and
establishing the presence of one or more sequences selected from the group consisting of Seq ID Nos. 1 to 6 or 10 in the genome of said celery plant.

19. A method for identifying a CeMV resistant celery plant, the method comprises the steps of:
isolating mRNA from said celery plant; and
establishing the absence of a cDNA represented by SEQ ID No. 8 and/or the presence of SEQ ID No.11 in said isolated mRNA.

20. A method for providing a CeMV resistant celery plant, the method comprising the step of introducing a mutated genomic region as defined in claim 1 into the genome of a celery plant.

21. (canceled)
